SEQUENCE LISTING (57 sequences)

SEQ ID NO: 1 - protein, 241 aa, Artificial Sequence
  Synthesized
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 2 - protein, 69 aa, Homo sapiens
TTTPAPRPPTPAPTIASQPLSLRPEACRPAAGGAVHTRGLDFACDIYIWAPLAGTCGVLLLSLVITLYC

SEQ ID NO: 3 - protein, 42 aa, Homo sapiens
KRGRKKLLYIFKQPFMRPVQTTQEEDGCSCRFPEEEEGGCEL

SEQ ID NO: 4 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 113 bp, Homo sapiens
cattttgacacccccataatatttttccagaattaacagtataaattgcatctcttgttcaagagttccctatcactctctttaatcactactcacagtaacctcaactcctg

SEQ ID NO: 6 - DNA, 277 bp, Homo sapiens
tcgaggtcgacggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccggga

SEQ ID NO: 7 - protein, 231 aa, Homo sapiens
MWGLKVLLLPVVSFARAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKM

SEQ ID NO: 8 - protein, 19 aa, Homo sapiens
ATNFSLLKQAGDVEENPGP

SEQ ID NO: 9 - DNA, 60 bp, Homo sapiens
atgtacaggatgcaactcctgtcttgcattgcactaagtcttgcacttgtcacgaattcg

SEQ ID NO: 10 - protein, 218 aa, Homo sapiens [sequence omitted]

SEQ ID NO: 11 - DNA, 714 bp, Artificial Sequence
  Synthesized
atcgtgggcggcaggagggccaggccccacgcctggcccttcatggtgagcctgcagctgaggggcggccacttctgcggcgccaccctgatcgcccccaacttcgtgatgagcgccgcccactgcgtggccaacgtgaacgtgagggccgtgagggtggtgctgggcgcccacaacctgagcaggagggagcccaccaggcaggtgttcgccgtgcagaggatcttcgagaacggctacgaccccgtgaacctgctgaacgacatcgtgatcctgcagctgaacggcagcgccaccatcaacgccaacgtgcaggtggcccagctgcccgcccagggcaggaggctgggcaacggcgtgcagtgcctggccatgggctggggcctgctgggcaggaacaggggcatcgccagcgtgctgcaggagctgaacgtgaccgtggtgaccagcctgtgcaggaggagcaacgtgtgcaccctggtgaggggcaggcaggccggcgtgtgcttcggcgacagcggcagccccctggtgtgcaacggcctgatccacggcatcgccagcttcgtgaggggcggctgcgccagcggcctgtaccccgacgccttcgcccccgtggcccagttcgtgaactggatcgacagcatcatccagatgtacaggatgcaactcctgtcttgcattgcactaagtcttgcacttgtcacgaattcg

SEQ ID NO: 12 - DNA, 714 bp, Artificial Sequence
  Synthesized
atgtacaggatgcaactcctgtcttgcattgcactaagtcttgcacttgtcacgaattcgatcgtgggcggcaggagggccaggccccacgcctggcccttcatggtgagcctgcagctgaggggcggccacttctgcggcgccaccctgatcgcccccaacttcgtgatgagcgccgcccactgcgtggccaacgtgaacgtgagggccgtgagggtggtgctgggcgcccacaacctgagcaggagggagcccaccaggcaggtgttcgccgtgcagaggatcttcgagaacggctacgaccccgtgaacctgctgaacgacatcgtgatcctgcagctgaacggcagcgccaccatcaacgccaacgtgcaggtggcccagctgcccgcccagggcaggaggctgggcaacggcgtgcagtgcctggccatgggctggggcctgctgggcaggaacaggggcatcgccagcgtgctgcaggagctgaacgtgaccgtggtgaccagcctgtgcaggaggagcaacgtgtgcaccctggtgaggggcaggcaggccggcgtgtgcttcggcgacagcggcagccccctggtgtgcaacggcctgatccacggcatcgccagcttcgtgaggggcggctgcgccagcggcctgtaccccgacgccttcgcccccgtggcccagttcgtgaactggatcgacagcatcatccag

SEQ ID NO: 13 - protein, 387 aa, Homo sapiens
MHPGVLAAFLFLSWTHCRAYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 14 - protein, 580 aa, Homo sapiens
MEALMARGALTGPLRALCLLGCLLSHAAAYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 15 - protein, 620 aa, Homo sapiens
MSLWQPLVLVLLVLGCCFAFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 16 - protein, 190 aa, Homo sapiens
MRLTVLCAVCLLPGSLAYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 17 - protein, 387 aa, Homo sapiens
MFSLKTLPFLLLLHVQISKALTPGNPKWERTNLTYRIRNYTPQLSEAEVERAIKDAFELWSVASPLIFTRISQGEADINIAFYQRDHGDNSPFDGPNGILAHAFQPGQGIGGDAHFDAEETWTNTSANYNLFLVAAHEFGHSLGLAHSSDPGALMYPNYAFRETSNYSLPQDDIDGIQAIYGLSSNPIQPTGPSTPKPCDPSLTFDAITTLRGEILFFKDRYFWRRHPQLQRVEMNFISLFWPSLPTGIQAAYEDFDRDLIFLFKGNQYWALSGYDILQGYPKDISNYGFPSSVQAIDAAVFYRSKTYFFVNDQFWRYDNQRQFMEPGYPKSISGAFPGIESKVDAVFQQEHFFHVFSGPRYYAFDLIAQRVTRVARGNKWLNCRYG

SEQ ID NO: 18 - protein, 395 aa, Homo sapiens
MKSLPILLLLCVAVCSAFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 19 - protein, 249 aa, Homo sapiens
MEHKEVVLLLLLFLKSGQGVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 20 - protein, 562 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 21 - protein, 431 aa, Homo sapiens
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 22 - protein, 339 aa, Homo sapiens
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 23 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 24 - protein, 476 aa, Homo sapiens
MMHLAFLVLLCLPVCSAYPLSGAAKEEDSNKDLAQQYLEKYYNLEKDVKQFRRKDSNLIVKKIQGMQKFLGLEVTGKLDTDTLEVMRKPRCGVPDVGHFSSFPGMPKWRKTHLTYRIVNYTPDLPRDAVDSAIEKALKVWEEVTPLTFSRLYEGEADIMISFAVKEHGDFYSFDGPGHSLAHAYPPGPGLYGDIHFDDDEKWTEDASGTNLFLVAAHELGHSLGLFHSANTEALMYPLYNSFTELAQFRLSQDDVNGIQSLYGPPPASTEEPLVPTKSVPSGSEMPAKCDPALSFDAISTLRGEYLFFKDRYFWRRSHWNPEPEFHLISAFWPSLPSYLDAAYEVNSRDTVFIFKGNEFWAIRGNEVQAGYPRGIHTLGFPPTIRKIDAAVSDKEKKKTYFFAADKYWRFDENSQSMEQGFPRLIADDFPGVEPKVDAVLQAFGFFYFFSGSSQFEFDPNARMVTHILKSNSWLHC

SEQ ID NO: 25 - protein, 476 aa, Homo sapiens; identical to SEQ ID NO: 24

SEQ ID NO: 26 - protein, 470 aa, Homo sapiens
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDVHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 27 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 28 - protein, 669 aa, Homo sapiens
MGSDPSAPGRPGWTGSLLGDREEAARPRLLPLLLVLLGCLGLGVAAEDAEVHAENWLRLYGYLPQPSRHMSTMRSAQILASALAEMQRFYGIPVTGVLDEETKEWMKRPRCGVPDQFGVRVKANLRRRRKRYALTGRKWNNHHLTFSIQNYTEKLGWYHSMEAVRRAFRVWEQATPLVFQEVPYEDIRLRRQKEADIMVLFASGFHGDSSPFDGTGGFLAHAYFPGPGLGGDTHFDADEPWTFSSTDLHGNNLFLVAVHELGHALGLEHSSNPNAIMAPFYQWKDVDNFKLPEDDLRGIQQLYGTPDGQPQPTQPLPTVTPRRPGRPDHRPPRPPQPPPPGGKPERPPKPGPPVQPRATERPDQYGPNICDGDFDTVAMLRGEMFVFKGRWFWRVRHNRVLDNYPMPIGHFWRGLPGDISAAYERQDGRFVFFKGDRYWLFREANLEPGYPQPLTSYGLGIPYDRIDTAIWWEPTGHTFFFQEDRYWRFNEETQRGDPGYPKPISVWQGIPASPKGAFLSNDAAYTYFYKGTKYWKFDNERLRMEPGYPKSILRDFMGCQEHVEPGPRWPDVARPPFNPHGGAEPGADSAEGDVGDGDGDFGAGVNKDGGSRVVVQMEEVARTVNVVMVLVPLLLLLCVLGLTYALVQMQRKGAPRVLLYCKRSLQEWV

SEQ ID NO: 29 - protein, 607 aa, Homo sapiens
MILLTFSTGRRLDFVHHSGVFFLQTLLWILCATVCGTEQYFNVEVWLQKYGYLPPTDPRMSVLRSAETMQSALAAMQQFYGINMTGKVDRNTIDWMKKPRCGVPDQTRGSSKFHIRRKRYALTGQKWQHKHITYSIKNVTPKVGDPETRKAIRRAFDVWQNVTPLTFEEVPYSELENGKRDVDITIIFASGFHGDSSPFDGEGGFLAHAYFPGPGIGGDTHFDSDEPWTLGNPNHDGNDLFLVAVHELGHALGLEHSNDPTAIMAPFYQYMETDNFKLPNDDLQGIQKIYGPPDKIPPPTRPLPTVPPHRSIPPADPRKNDRPKPPRPPTGRPSYPGAKPNICDGNFNTLAILRREMFVFKDQWFWRVRNNRVMDGYPMQITYFWRGLPPSIDAVYENSDGNFVFFKGNKYWVFKDTTLQPGYPHDLITLGSGIPPHGIDSAIWWEDVGKTYFFKGDRYWRYSEEMKTMDPGYPKPITVWKGIPESPQGAFVHKENGFTYFYKGKEYWKFNNQILKVEPGYPRSILKDFMGCDGPTDRVKEGHSPPDDVDIVIKLDNTASTVKAIAIVIPCILALCLLVLVYTVFQFKRKGTPRHILYCKRSMQEWV

SEQ ID NO: 30 - protein, 603 aa, Homo sapiens
MRRRAARGPGPPPPGPGLSRLPLPLLLLLALGTRGGCAAPAPAPRAEDLSLGVEWLSRFGYLPPADPTTGQLQTQEELSKAITAMQQFGGLEATGILDEATLALMKTPRCSLPDLPVLTQARRRRQAPAPTKWNKRNLSWRVRTFPRDSPLGHDTVRALMYYALKVWSDIAPLNFHEVAGSAADIQIDFSKADHNDGYPFDGPGGTVAHAFFPGHHHTAGDTHFDDDEAWTFRSSDAHGMDLFAVAVHEFGHAIGLSHVAAAHSIMRPYYQGPVGDPLRYGLPYEDKVRVWQLYGVRESVSPTAQPEEPPLLPEPPDNRSSAPPRKDVPHRCSTHFDAVAQIRGEAFFFKGKYFWRLTRDRHLVSLQPAQMHRFWRGLPLHLDSVDAVYERTSDHKIVFFKGDRYWVFKDNNVEEGYPRPVSDFSLPPGGIDAAFSWAHNDRTYFFKDQLYWRYDDHTRHMDPGYPAQSPLWRGVPSTLDDAMRWSDGASYFFRGQEYWKVLDGELEVAPGYPQSTARDWLVCGDSQADGSVAAGVDAAEGPRAPPGQHDQSRSEDGYEVCSCTSGASSPPGAPGPLVAATMLLLLPPLSPGALWTAAQALTL

SEQ ID NO: 31 - protein, 645 aa, Homo sapiens
MPRSRGGRAAPGPPPPPPPPGQAPRWSRWRVPGRLLLLLLPALCCLPGAARAAAAAAGAGNRAAVAVAVARADEAEAPFAGQNWLKSYGYLLPYDSRASALHSAKALQSAVSTMQQFYGIPVTGVLDQTTIEWMKKPRCGVPDHPHLSRRRRNKRYALTGQKWRQKHITYSIHNYTPKVGELDTRKAIRQAFDVWQKVTPLTFEEVPYHEIKSDRKEADIMIFFASGFHGDSSPFDGEGGFLAHAYFPGPGIGGDTHFDSDEPWTLGNANHDGNDLFLVAVHELGHALGLEHSSDPSAIMAPFYQYMETHNFKLPQDDLQGIQKIYGPPAEPLEPTRPLPTLPVRRIHSPSERKHERQPRPPRPPLGDRPSTPGTKPNICDGNFNTVALFRGEMFVFKDRWFWRLRNNRVQEGYPMQIEQFWKGLPARIDAAYERADGRFVFFKGDKYWVFKEVTVEPGYPHSLGELGSCLPREGIDTALRWEPVGKTYFFKGERYWRYSEERRATDPGYPKPITVWKGIPQAPQGAFISKEGYYTYFYKGRDYWKFDNQKLSVEPGYPRNILRDWMGCNQKEVERRKERRLPQDDVDIMVTINDVPGSVNAVAVVIPCILSLCILVLVYTIFQFKNKTGPQPVTYYKRPVQEWV

SEQ ID NO: 32 - protein, 562 aa, Homo sapiens
MRLRLRLLALLLLLLAPPARAPKPSAQDVSLGVDWLTRYGYLPPPHPAQAQLQSPEKLRDAIKVMQRFAGLPETGRMDPGTVATMRKPRCSLPDVLGVAGLVRRRRRYALSGSVWKKRTLTWRVRSFPQSSQLSQETVRVLMSYALMAWGMESGLTFHEVDSPQGQEPDILIDFARAFHQDSYPFDGLGGTLAHAFFPGEHPISGDTHFDDEETWTFGSKDGEGTDLFAVAVHEFGHALGLGHSSAPNSIMRPFYQGPVGDPDKYRLSQDDRDGLQQLYGKAPQTPYDKPTRKPLAPPPQPPASPTHSPSFPIPDRCEGNFDAIANIRGETFFFKGPWFWRLQPSGQLVSPRPARLHRFWEGLPAQVRVVQAAYARHRDGRILLFSGPQFWVFQDRQLEGGARPLTELGLPPGEEVDAVFSWPQNGKTYLVRGRQYWRYDEAAARPDPGYPRDLSLWEGAPPSPDDVTVSNAGDTYFFKGAHYWRFPKNSIKTEPDAPQPMGPNWLDCPAPSSGPRAPRPPKATPVSETCDCQCELNQAAGRWPAPIPLLLLPLLVGGVASR

SEQ ID NO: 33 - protein, 261 aa, Homo sapiens
MQLVILRVTIFLPWCFAVPVPPAADHKGWDFVEGYFHQFFLTKKESPLLTQETQTQLLQQFHRNGTDLLDMQMHALLHQPHCGVPDGSDTSISPGRCKWNKHTLTYRIINYPHDMKPSAVKDSIYNAVSIWSNVTPLIFQQVQNGDADIKVSFWQWAHEDGWPFDGPGGILGHAFLPNSGNPGVVHFDKNEHWSASDTGYNLFLVATHEIGHSLGLQHSGNQSSIMYPTYWYHDPRTFQLSADDIQRIQHLYGEKCSSDIP

SEQ ID NO: 34 - protein, 967 aa, Homo sapiens
MQRAVPEGFGRRKLGSDMGNAERAPGSRSFGPVPTLLLLAAALLAVSDALGRPSEEDEELVVPELERAPGHGTTRLRLHAFDQQLDLELRPDSSFLAPGFTLQNVGRKSGSETPLPETDLAHCFYSGTVNGDPSSAAALSLCEGVRGAFYLLGEAYFIQPLPAASERLATAAPGEKPPAPLQFHLLRRNRQGDVGGTCGVVDDEPRPTGKAETEDEDEGTEGEDEGAQWSPQDPALQGVGQPTGTGSIRKKRFVSSHRYVETMLVADQSMAEFHGSGLKHYLLTLFSVAARLYKHPSIRNSVSLVVVKILVIHDEQKGPEVTSNAALTLRNFCNWQKQHNPPSDRDAEHYDTAILFTRQDLCGSQTCDTLGMADVGTVCDPSRSCSVIEDDGLQAAFTTAHELGHVFNMPHDDAKQCASLNGVNQDSHMMASMLSNLDHSQPWSPCSAYMITSFLDNGHGECLMDKPQNPIQLPGDLPGTSYDANRQCQFTFGEDSKHCPDAASTCSTLWCTGTSGGVLVCQTKHFPWADGTSCGEGKWCINGKCVNKTDRKHFDTPFHGSWGMWGPWGDCSRTCGGGVQYTMRECDNPVPKNGGKYCEGKRVRYRSCNLEDCPDNNGKTFREEQCEAHNEFSKASFGSGPAVEWIPKYAGVSPKDRCKLICQAKGIGYFFVLQPKVVDGTPCSPDSTSVCVQGQCVKAGCDRIIDSKKKFDKCGVCGGNGSTCKKISGSVTSAKPGYHDIITIPTGATNIEVKQRNQRGSRNNGSFLAIKAADGTYILNGDYTLSTLEQDIMYKGVVLRYSGSSAALERIRSFSPLKEPLTIQVLTVGNALRPKIKYTYFVKKKKESFNAIPTFSAWVIEEWGECSKSCELGWQRRLVECRDINGQPASECAKEVKPASTRPCADHPCPQWQLGEWSSCSKTCGKGYKKRSLKCLSHDGGVLSHESCDPLKKPKHFIDFCTMAECS

SEQ ID NO: 35 - protein, 837 aa, Homo sapiens
MSQTGSHPGRGLAGRWLWGAQPCLLLPIVPLSWLVWLLLLLLASLLPSARLASPLPREEEIVFPEKLNGSVLPGSGAPARLLCRLQAFGETLLLELEQDSGVQVEGLTVQYLGQAPELLGGAEPGTYLTGTINGDPESVASLHWDGGALLGVLQYRGAELHLQPLEGGTPNSAGGPGAHILRRKSPASGQGPMCNVKAPLGSPSPRPRRAKRFASLSRFVETLVVADDKMAAFHGAGLKRYLLTVMAAAAKAFKHPSIRNPVSLVVTRLVILGSGEEGPQVGPSAAQTLRSFCAWQRGLNTPEDSDPDHFDTAILFTRQDLCGVSTCDTLGMADVGTVCDPARSCAIVEDDGLQSAFTAAHELGHVFNMLHDNSKPCISLNGPLSTSRHVMAPVMAHVDPEEPWSPCSARFITDFLDNGYGHCLLDKPEAPLHLPVTFPGKDYDADRQCQLTFGPDSRHCPQLPPPCAALWCSGHLNGHAMCQTKHSPWADGTPCGPAQACMGGRCLHMDQLQDFNIPQAGGWGPWGPWGDCSRTCGGGVQFSSRDCTRPVPRNGGKYCEGRRTRFRSCNTEDCPTGSALTFREEQCAAYNHRTDLFKSFPGPMDWVPRYTGVAPQDQCKLTCQAQALGYYYVLEPRVVDGTPCSPDSSSVCVQGRCIHAGCDRIIGSKKKFDKCMVCGGDGSGCSKQSGSFRKFRYGYNNVVTIPAGATHILVRQQGNPGHRSIYLALKLPDGSYALNGEYTLMPSPTDVVLPGAVSLRYSGATAASETLSGHGPLAQPLTLQVLVAGNPQDTRLRYSFFVPRPTPSTPRPTPQDWLHRRAQILEILRRRPWAGRK

SEQ ID NO: 36 - protein, 930 aa, Homo sapiens
MLLGWASLLLCAFRLPLAAVGPAATPAQDKAGQPPTAAAAAQPRRRQGEEVQERAEPPGHPHPLAQRRRSKGLVQNIDQLYSGGGKVGYLVYAGGRRFLLDLERDGSVGIAGFVPAGGGTSAPWRHRSHCFYRGTVDGSPRSLAVFDLCGGLDGFFAVKHARYTLKPLLRGPWAEEEKGRVYGDGSARILHVYTREGFSFEALPPRASCETPASTPEAHEHAPAHSNPSGRAALASQLLDQSALSPAGGSGPQTWWRRRRRSISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSLMPCPPNGKSFRHEQCEAKNGYQSDAKGVKTFVEWVPKYAGVLPADVCKLTCRAKGTGYYVVFSPKVTDGTECRLYSNSVCVRGKCVRTGCDGIIGSKLQYDKCGVCGGDNSSCTKIVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKPLDVRYSFFVPKKSTPKVNSVTSHGSNKVGSHTSQPQWVTGPWLACSRTCDTGWHTRTVQCQDGNRKLAKGCPLSQRPSAFKQCLLKKC

SEQ ID NO: 37 - protein, 889 aa, Homo sapiens
MLPAPAAPRWPPLLLLLLLLLPLARGAPARPAAGGQASELVVPTRLPGSAGELALHLSAFGKGFVLRLAPDDSFLAPEFKIERLGGSGRATGGERGLRGCFFSGTVNGEPESLAAVSLCRGLSGSFLLDGEEFTIQPQGAGGSLAQPHRLQRWGPAGARPLPRGPEWEVETGEGQRQERGDHQEDSEEESQEEEAEGASEPPPPLGATSRTKRFVSEARFVETLLVADASMAAFYGADLQNHILTLMSVAARIYKHPSIKNSINLMVVKVLIVEDEKWGPEVSDNGGLTLRNFCNWQRRFNQPSDRHPEHYDTAILLTRQNFCGQEGLCDTLGVADIGTICDPNKSCSVIEDEGLQAAHTLAHELGHVLSMPHDDSKPCTRLFGPMGKHHVMAPLFVHLNQTLPWSPCSAMYLTELLDGGHGDCLLDAPAAALPLPTGLPGRMALYQLDQQCRQIFGPDFRHCPNTSAQDVCAQLWCHTDGAEPLCHTKNGSLPWADGTPCGPGHLCSEGSCLPEEEVERPKPVADGGWAPWGPWGECSRTCGGGVQFSHRECKDPEPQNGGRYCLGRRAKYQSCHTEECPPDGKSFREQQCEKYNAYNYTDMDGNLLQWVPKYAGVSPRDRCKLFCRARGRSEFKVFEAKVIDGTLCGPETLAICVRGQCVKAGCDHVVDSPRKLDKCGVCGGKGNSCRKVSGSLTPTNYGYNDIVTIPAGATNIDVKQRSHPGVQNDGNYLALKTADGQYLLNGNLAISAIEQDILVKGTILKYSGSIATLERLQSFRPLPEPLTVQLLTVPGEVFPPKVKYTFFVPNDVDFSMQSSKERATTNIIQPLLHAQWVLGDWSECSSTCGAGWQRRTVECRDPSGQASATCNKALKPEDAKPCESQLCPL

SEQ ID NO: 38 - protein, 1935 aa, Homo sapiens
MQFVSWATLLTLLVRDLAEMGSPDAAAAVRKDRLHPRQVKLLETLSEYEIVSPIRVNALGEPFPTNVHFKRTRRSINSATDPWPAFASSSSSSTSSQAHYRLSAFGQQFLFNLTANAGFIAPLFTVTLLGTPGVNQTKFYSEEEAELKHCFYKGYVNTNSEHTAVISLCSGMLGTFRSHDGDYFIEPLQSMDEQEDEEEQNKPHIIYRRSAPQREPSTGRHACDTSEHKNRHSKDKKKTRARKWGERINLAGDVAALNSGLATEAFSAYGNKTDNTREKRTHRRTKRFLSYPRFVEVLVVADNRMVSYHGENLQHYILTLMSIVASIYKDPSIGNLINIVIVNLIVIHNEQDGPSISFNAQTTLKNFCQWQHSKNSPGGIHHDTAVLLTRQDICRAHDKCDTLGLAELGTICDPYRSCSISEDSGLSTAFTIAHELGHVFNMPHDDNNKCKEEGVKSPQHVMAPTLNFYTNPWMWSKCSRKYITEFLDTGYGECLLNEPESRPYPLPVQLPGILYNVNKQCELIFGPGSQVCPYMMQCRRLWCNNVNGVHKGCRTQHTPWADGTECEPGKHCKYGFCVPKEMDVPVTDGSWGSWSPFGTCSRTCGGGIKTAIRECNRPEPKNGGKYCVGRRMKFKSCNTEPCLKQKRDFRDEQCAHFDGKHFNINGLLPNVRWVPKYSGILMKDRCKLFCRVAGNTAYYQLRDRVIDGTPCGQDTNDICVQGLCRQAGCDHVLNSKARRDKCGVCGGDNSSCKTVAGTFNTVHYGYNTVVRIPAGATNIDVRQHSFSGETDDDNYLALSSSKGEFLLNGNFVVTMAKREIRIGNAVVEYSGSETAVERINSTDRIEQELLLQVLSVGKLYNPDVRYSFNIPIEDKPQQFYWNSHGPWQACSKPCQGERKRKLVCTRESDQLTVSDQRCDRLPQPGHITEPCGTDCDLRWHVASRSECSAQCGLGYRTLDIYCAKYSRLDGKTEKVDDGFCSSHPKPSNREKCSGECNTGGWRYSAWTECSKSCDGGTQRRRAICVNTRNDVLDDSKCTHQEKVTIQRCSEFPCPQWKSGDWSECLVTCGKGHKHRQVWCQFGEDRLNDRMCDPETKPTSMQTCQQPECASWQAGPWGQCSVTCGQGYQLRAVKCIIGTYMSVVDDNDCNAATRPTDTQDCELPSCHPPPAAPETRRSTYSAPRTQWRFGSWTPCSATCGKGTRMRYVSCRDENGSVADESACATLPRPVAKEECSVTPCGQWKALDWSSCSVTCGQGRATRQVMCVNYSDHVIDRSECDQDYIPETDQDCSMSPCPQRTPDSGLAQHPFQNEDYRPRSASPSRTHVLGGNQWRTGPWGACSSTCAGGSQRRVVVCQDENGYTANDCVERIKPDEQRACESGPCPQWAYGNWGECTKLCGGGIRTRLVVCQRSNGERFPDLSCEILDKPPDREQCNTHACPHDAAWSTGPWSSCSVSCGRGHKQRNVYCMAKDGSHLESDYCKHLAKPHGHRKCRGGRCPKWKAGAWSQCSVSCGRGVQQRHVGCQIGTHKIARETECNPYTRPESERDCQGPRCPLYTWRAEEWQECTKTCGEGSRYRKVVCVDDNKNEVHGARCDVSKRPVDRESCSLQPCEYVWITGEWSECSVTCGKGYKQRLVSCSEIYTGKENYEYSYQTTINCPGTQPPSVHPCYLRDCPVSATWRVGNWGSCSVSCGVGVMQRSVQCLTNEDQPSHLCHTDLKPEERKTCRNVYNCELPQNCKEVKRLKGASEDGEYFLMIRGKLLKIFCAGMHSDHPKEYVTLVHGDSENFSEVYGHRLHNPTECPYNGSRRDDCQCRKDYTAAGFSSFQKIRIDLTSMQIITTDLQFARTSEGHPVPFATAGDCYSAAKCPQGRFSINLYGTGLSLTESARWISQGNYAVSDIKKSPDGTRVVGKCGGYCGKCTPSSGTGLEVRVL

SEQ ID NO: 39 - protein, 950 aa, Homo sapiens
MLLLGILTLAFAGRTAGGSEPEREVVVPIRLDPDINGRRYYWRGPEDSGDQGLIFQITAFQEDFYLHLTPDAQFLAPAFSTEHLGVPLQGLTGGSSDLRRCFYSGDVNAEPDSFAAVSLCGGLRGAFGYRGAEYVISPLPNASAPAAQRNSQGAHLLQRRGVPGGPSGDPTSRCGVASGWNPAILRALDPYKPRRAGFGESRSRRRSGRAKRFVSIPRYVETLVVADESMVKFHGADLEHYLLTLLATAARLYRHPSILNPINIVVVKVLLLRDRDSGPKVTGNAALTLRNFCAWQKKLNKVSDKHPEYWDTAILFTRQDLCGATTCDTLGMADVGTMCDPKRSCSVIEDDGLPSAFTTAHELGHVFNMPHDNVKVCEEVFGKLRANHMMSPTLIQIDRANPWSACSAAIITDFLDSGHGDCLLDQPSKPISLPEDLPGASYTLSQQCELAFGVGSKPCPYMQYCTKLWCTGKAKGQMVCQTRHFPWADGTSCGEGKLCLKGACVERHNLNKHRVDGSWAKWDPYGPCSRTCGGGVQLARRQCTNPTPANGGKYCEGVRVKYRSCNLEPCPSSASGKSFREEQCEAFNGYNHSTNRLTLAVAWVPKYSGVSPRDKCKLICRANGTGYFYVLAPKVVDGTLCSPDSTSVCVQGKCIKAGCDGNLGSKKRFDKCGVCGGDNKSCKKVTGLFTKPMHGYNFVVAIPAGASSIDIRQRGYKGLIGDDNYLALKNSQGKYLLNGHFVVSAVERDLVVKGSLLRYSGTGTAVESLQASRPILEPLTVEVLSVGKMTPPRVRYSFYLPKEPREDKSSHPKDPRGPSVLHNSVLSLSNQVEQPDDRPPARWVAGSWGPCSASCGSGLQKRAVDCRGSAGQRTVPACDAAHRPVETQACGEPCPTWELSAWSPCSKSCGRGFQRRSLKCVGHGGRLLARDQCNLHRKPQELDFCVLRPC

SEQ ID NO: 40 - protein, 1910 aa, Homo sapiens
MWVAKWLTGLLYHLSLFITRSWEVDFHPRQEALVRTLTSYEVVIPERVNEFGEVFPQSHHFSRQKRSSEALEPMPFRTHYRFTAYGQLFQLNLTADASFLAAGYTEVHLGTPERGAWESDAGPSDLRHCFYRGQVNSQEDYKAVVSLCGGLTGTFKGQNGEYFLEPIMKADGNEYEDGHNKPHLIYRQDLNNSFLQTLKYCSVSESQIKETSLPFHTYSNMNEDLNVMKERVLGHTSKNVPLKDERRHSRKKRLISYPRYIEIMVTADAKVVSAHGSNLQNYILTLMSIVATIYKDPSIGNLIHIVVVKLVMIHREEEGPVINFDGATTLKNFCSWQQTQNDLDDVHPSHHDTAVLITREDICSSKEKCNMLGLSYLGTICDPLQSCFINEEKGLISAFTIAHELGHTLGVQHDDNPRCKEMKVTKYHVMAPALSFHMSPWSWSNCSRKYVTEFLDTGYGECLLDKPDEEIYNLPSELPGSRYDGNKQCELAFGPGSQMCPHINICMHLWCTSTEKLHKGCFTQHVPPADGTDCGPGMHCRHGLCVNKETETRPVNGEWGPWEPYSSCSRTCGGGIESATRRCNRPEPRNGGNYCVGRRMKFRSCNTDSCPKGTQDFREKQCSDFNGKHLDISGIPSNVRWLPRYSGIGTKDRCKLYCQVAGTNYFYLLKDMVEDGTPCGTETHDICVQGQCMAAGCDHVLNSSAKIDKCGVCGGDNSSCKTITGVFNSSHYGYNVVVKIPAGATNVDIRQYSYSGQPDDSYLALSDAEGNFLFNGNFLLSTSKKEINVQGTRTVIEYSGSNNAVERINSTNRQEKEILIEVLCVGNLYNPDVHYSFNIPLEERSDMFTWDPYGPWEGCTKMCQGLQRRNITCIHKSDHSVVSDKECDHLPLPSFVTQSCNTDCELRWHVIGKSECSSQCGQGYRTLDIHCMKYSIHEGQTVQVDDHYCGDQLKPPTQELCHGNCVFTRWHYSEWSQCSRSCGGGERSRESYCMNNFGHRLADNECQELSRVTRENCNEFSCPSWAASEWSECLVTCGKGTKQRQVWCQLNVDHLSDGFCNSSTKPESLSPCELHTCASWQVGPWGPCTTTCGHGYQMRDVKCVNELASAVLEDTECHEASRPSDRQSCVLTPCSFISKLETALLPTVLIKKMAQWRHGSWTPCSVSCGRGTQARYVSCRDALDRIADESYCAHLPRPAEIWDCFTPCGEWQAGDWSPCSASCGHGKTTRQVLCMNYHQPIDENYCDPEVRPLMEQECSLAACPPAHSHFPSSPVQPSYYLSTNLPLTQKLEDNENQVVHPSVRGNQWRTGPWGSCSSSCSGGLQHRAVVCQDENGQSASYCDAASKPPELQQCGPGPCPQWNYGNWGECSQTCGGGIKSRLVICQFPNGQILEDHNCEIVNKPPSVIQCHMHACPADVSWHQEPWTSCSASCGKGRKYREVFCIDQFQRKLEDTNCSQVQKPPTHKACRSVRCPSWKANSWNECSVTCGSGVQQRDVYCRLKGVGQVVEEMCDQSTRPCSQRRCWSQDCVQHKGMERGRLNCSTSCERKDSHQRMECTDNQIRQVNEIVYNSSTISLTSKNCRNPPCNYIVVTADSSQCANNCGFSYRQRITYCTEIPSTKKHKLHRLRPIVYQECPVVPSSQVYQCINSCLHLATWKVGKWSKCSVTCGIGIMKRQVKCITKHGLSSDLCLNHLKPGAQKKCYANDCKSFTTCKEIQVKNHIRKDGDYYLNIKGRIIKIYCADMYLENPKEYLTLVQGEENFSEVYGFRLKNPYQCPFNGSRREDCECDNGHLAAGYTVFSKIRIDLTSMQIKTTDLLFSKTIFGNAVPFATAGDCYSAFRCPQGQFSINLSGTGMKISSTAKWLTQGSYTSVSIRRSEDGTRFFGKCGGYCGKCLPHMTTGLPIQVI

SEQ ID NO: 41 - protein, 543 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATKEDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 42 - protein, 20 aa, Homo sapiens
MYRMQLLSCIALSLALVTNS

SEQ ID NO: 43 - protein, 23 aa, Homo sapiens
MYRMQLLSCIALSLALVTNSAAS

SEQ ID NO: 44 - protein, 22 aa, Artificial Sequence
  Synthesized
MDAMKRGLCCVLLLCGAVFVSA

SEQ ID NO: 45 - protein, 23 aa, Artificial Sequence
  Synthesized
MDAMKRGLCCVLLLCGAVFVSAS

SEQ ID NO: 46 - protein, 27 aa, Homo sapiens
MTLGRRLACLFLACVLPALLLGGTALA

SEQ ID NO: 47 - protein, 633 aa, Homo sapiens
MSLWQPLVLVLLVLGCCFAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 48 - DNA, 1089 bp, Artificial Sequence
  Synthesized
tcgaggtcgacggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctggccaccatgtggggactgaaggtgctgctgctgcccgtggtgagctttgccagagcccccgacagcgtggactatagaaagaagggctacgtcacccccgtgaagaatcaaggccagtgcggcagctgctgggcctttagctccgtgggcgctctggagggacagctgaagaagaagaccggcaagctgctcaatctgagcccccaaaacctcgtggactgcgtgagcgagaatgatggctgtggaggcggctatatgaccaacgccttccagtacgtgcagaagaatagaggcatcgacagcgaagacgcctacccctacgtgggccaagaggagagctgcatgtacaatcccaccggcaaggctgccaaatgtagaggctacagagagatccccgagggcaacgagaaggctctgaagagggctgtcgctagggtgggacccgtgagcgtcgccatcgatgcctctctgacaagcttccagttctacagcaagggcgtgtactacgacgagagctgcaacagcgacaatctgaaccacgccgtgctcgctgtgggatacggcatccagaagggcaacaagcactggatcatcaagaacagctggggcgagaactggggcaacaagggctacattctgatggctagaaacaagaacaacgcttgcggcatcgccaatctggccagcttccccaagatg

SEQ ID NO: 49 - protein, 450 aa, Artificial Sequence
  Synthesized
MYRMQLLSCIALSLALVTNSIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQGSGATNFSLLKQAGDVEENPGPMRLTVLCAVCLLPGSLAYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 50 - DNA, 1350 bp, Artificial Sequence
  Synthesized
atgtacaggatgcaactcctgtcttgcattgcactaagtcttgcacttgtcacgaattcgatcgtgggaggcagaagagccagaccccacgcttggcccttcatggtgtctctgcagctgagaggcggccacttctgtggcgccacactgatcgcccccaacttcgtgatgagcgccgcccactgcgtggccaatgtgaatgtgagagccgtgagggtggtgctgggagcccacaatctgtctaggagagagcctacaaggcaagtgttcgccgtccagaggatcttcgagaacggctacgaccccgtgaatctgctgaacgacatcgtcattctgcagctgaacggcagcgctaccatcaacgccaatgtgcaagtggcccagctgcccgcccaaggcagaagactgggcaacggcgtgcagtgtctggctatgggatggggactgctgggcagaaacagaggaattgccagcgtgctccaagagctgaacgtgacagtggtgacatctctgtgtagaagaagcaacgtgtgcacactggtgagaggcagacaagccggagtgtgcttcggcgacagcggcagccctctggtgtgcaatggactgatccacggcatcgccagcttcgtgagaggaggctgtgccagcggactgtatcccgacgccttcgcccccgtcgcccagttcgtcaactggatcgactccatcatccagggttccggagctaccaacttttccctcctcaagcaagccggcgatgtcgaggagaatcccggacccatgaggctcacagtgctgtgtgccgtgtgtctgctgcccggctctctggcttactctctgttccccaacagccccaagtggaccagcaaagtggtgacctatagaatcgtgagctacaccagagatctgccccacatcaccgtggacagactggtgagcaaggctctgaacatgtggggcaaggagatccctctgcactttaggaaggtggtgtggggcaccgccgacatcatgatcggattcgccagaggcgctcacggagacagctaccctttcgatggccccggaaatacactggcccacgcctttgcccccggcaccggactgggaggagatgcccactttgacgaggacgagaggtggacagacggctcctctctgggcatcaactttctgtacgctgccacccatgagctgggacacagcctcggcatgggacacagcagcgaccccaacgccgtgatgtaccctacctatggcaatggcgatccccagaacttcaagctgtcccaagacgacatcaagggcatccagaagctgtacggaaagaggtccaactctaggaagaag

SEQ ID NO: 51 - protein, 491 aa, Artificial Sequence
  Synthesized
MWGLKVLLLPVVSFARAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKMGSGATNFSLLKQAGDVEENPGPMYRMQLLSCIALSLALVTNSIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQ

SEQ ID NO: 52 - DNA, 1869 bp, Artificial Sequence
  Synthesized
tcgaggtcgacggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctggccaccatgtggggactgaaggtgctgctgctgcccgtggtgagctttgccagagcccccgacagcgtggactatagaaagaagggctacgtcacccccgtgaagaatcaaggccagtgcggcagctgctgggcctttagctccgtgggcgctctggagggacagctgaagaagaagaccggcaagctgctcaatctgagcccccaaaacctcgtggactgcgtgagcgagaatgatggctgtggaggcggctatatgaccaacgccttccagtacgtgcagaagaatagaggcatcgacagcgaagacgcctacccctacgtgggccaagaggagagctgcatgtacaatcccaccggcaaggctgccaaatgtagaggctacagagagatccccgagggcaacgagaaggctctgaagagggctgtcgctagggtgggacccgtgagcgtcgccatcgatgcctctctgacaagcttccagttctacagcaagggcgtgtactacgacgagagctgcaacagcgacaatctgaaccacgccgtgctcgctgtgggatacggcatccagaagggcaacaagcactggatcatcaagaacagctggggcgagaactggggcaacaagggctacattctgatggctagaaacaagaacaacgcttgcggcatcgccaatctggccagcttccccaagatgggttccggagctaccaacttttccctcctcaagcaagccggcgatgtcgaggagaatcccggacccatgtacaggatgcaactcctgtcttgcattgcactaagtcttgcacttgtcacgaattcgatcgtgggaggcagaagagccagaccccacgcttggcccttcatggtgtctctgcagctgagaggcggccacttctgtggcgccacactgatcgcccccaacttcgtgatgagcgccgcccactgcgtggccaatgtgaatgtgagagccgtgagggtggtgctgggagcccacaatctgtctaggagagagcctacaaggcaagtgttcgccgtccagaggatcttcgagaacggctacgaccccgtgaatctgctgaacgacatcgtcattctgcagctgaacggcagcgctaccatcaacgccaatgtgcaagtggcccagctgcccgcccaaggcagaagactgggcaacggcgtgcagtgtctggctatgggatggggactgctgggcagaaacagaggaattgccagcgtgctccaagagctgaacgtgacagtggtgacatctctgtgtagaagaagcaacgtgtgcacactggtgagaggcagacaagccggagtgtgcttcggcgacagcggcagccctctggtgtgcaatggactgatccacggcatcgccagcttcgtgagaggaggctgtgccagcggactgtatcccgacgccttcgcccccgtcgcccagttcgtcaactggatcgactccatcatccag

SEQ ID NO: 53 - protein, 703 aa, Artificial Sequence
  Synthesized
MWGLKVLLLPVVSFARAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKMGSGATNFSLLKQAGDVEENPGPMYRMQLLSCIALSLALVTNSIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQGSGATNFSLLKQAGDVEENPGPMRLTVLCAVCLLPGSLAYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 54 - DNA, 2505 bp, Artificial Sequence
  Synthesized
tcgaggtcgacggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctggccaccatgtggggactgaaggtgctgctgctgcccgtggtgagctttgccagagcccccgacagcgtggactatagaaagaagggctacgtcacccccgtgaagaatcaaggccagtgcggcagctgctgggcctttagctccgtgggcgctctggagggacagctgaagaagaagaccggcaagctgctcaatctgagcccccaaaacctcgtggactgcgtgagcgagaatgatggctgtggaggcggctatatgaccaacgccttccagtacgtgcagaagaatagaggcatcgacagcgaagacgcctacccctacgtgggccaagaggagagctgcatgtacaatcccaccggcaaggctgccaaatgtagaggctacagagagatccccgagggcaacgagaaggctctgaagagggctgtcgctagggtgggacccgtgagcgtcgccatcgatgcctctctgacaagcttccagttctacagcaagggcgtgtactacgacgagagctgcaacagcgacaatctgaaccacgccgtgctcgctgtgggatacggcatccagaagggcaacaagcactggatcatcaagaacagctggggcgagaactggggcaacaagggctacattctgatggctagaaacaagaacaacgcttgcggcatcgccaatctggccagcttccccaagatgggttccggagctaccaacttttccctcctcaagcaagccggcgatgtcgaggagaatcccggacccatgtacaggatgcaactcctgtcttgcattgcactaagtcttgcacttgtcacgaattcgatcgtgggaggcagaagagccagaccccacgcttggcccttcatggtgtctctgcagctgagaggcggccacttctgtggcgccacactgatcgcccccaacttcgtgatgagcgccgcccactgcgtggccaatgtgaatgtgagagccgtgagggtggtgctgggagcccacaatctgtctaggagagagcctacaaggcaagtgttcgccgtccagaggatcttcgagaacggctacgaccccgtgaatctgctgaacgacatcgtcattctgcagctgaacggcagcgctaccatcaacgccaatgtgcaagtggcccagctgcccgcccaaggcagaagactgggcaacggcgtgcagtgtctggctatgggatggggactgctgggcagaaacagaggaattgccagcgtgctccaagagctgaacgtgacagtggtgacatctctgtgtagaagaagcaacgtgtgcacactggtgagaggcagacaagccggagtgtgcttcggcgacagcggcagccctctggtgtgcaatggactgatccacggcatcgccagcttcgtgagaggaggctgtgccagcggactgtatcccgacgccttcgcccccgtcgcccagttcgtcaactggatcgactccatcatccagggttccggagctaccaacttttccctcctcaagcaagccggcgatgtcgaggagaatcccggacccatgaggctcacagtgctgtgtgccgtgtgtctgctgcccggctctctggcttactctctgttccccaacagccccaagtggaccagcaaagtggtgacctatagaatcgtgagctacaccagagatctgccccacatcaccgtggacagactggtgagcaaggctctgaacatgtggggcaaggagatccctctgcactttaggaaggtggtgtggggcaccgccgacatcatgatcggattcgccagaggcgctcacggagacagctaccctttcgatggccccggaaatacactggcccacgcctttgcccccggcaccggactgggaggagatgcccactttgacgaggacgagaggtggacagacggctcctctctgggcatcaactttctgtacgctgccacccatgagctgggacacagcctcggcatgggacacagcagcgaccccaacgccgtgatgtaccctacctatggcaatggcgatccccagaacttcaagctgtcccaagacgacatcaagggcatccagaagctgtacggaaagaggtccaactctaggaagaag

SEQ ID NO: 55 - protein, 54 aa, Artificial Sequence
  Synthesized
LDLEMLAPYIPMDDDFQLLDLEMLAPYIPMDDDFQLLDLEMLAPYIPMDDDFQL

SEQ ID NO: 56 - protein, 36 aa, Artificial Sequence
  Synthesized
LDLEMLAPYIPMDDDFQLLDLEMLAPYIPMDDDFQL

SEQ ID NO: 57 - protein, 18 aa, Homo sapiens
LDLEMLAPYIPMDDDFQL

(19) United States
(12) Patent Application Publication
(10) Pub. No.: US 2024/0307442 A1
(43) Pub. Date: Sep. 19, 2024

(54) Modified Cell Targeting Tumor ECM and Cellular Therapy thereof

(71) Applicants: Innovative Cellular Therapeutics Holdings, Ltd., George Town, Grand Cayman (KY); Innovative Cellular Therapeutics, Inc., Rockville, MD (US)

(72) Inventors: Wei DING, Shanghai (CN); Yang ZHAO, Shanghai (CN); Guiting HAN, Shanghai (CN); Yuzhe PENG, Shanghai (CN); Xianyang JIANG, Shanghai (CN); Zhiyuan CAO, Shanghai (CN); Lei XIAO, Rockville, MD (US); Le TIAN, Rockville, MD (US)

(73) Assignees: Innovative Cellular Therapeutics Holdings, Ltd., George Town, Grand Cayman (KY); Innovative Cellular Therapeutics, Inc., Rockville, MD (US)

(21) Appl. No.: 18/571,062

(22) PCT Filed: Jun. 16, 2022
(86) PCT No.: PCT/US2022/072981

§ 371 Date: Dec. 15, 2023

Related U.S. Application Data
(60) Provisional application No. 63/218,020, filed on Jul. 2, 2021.
(60) Provisional application No. 63/212,303, filed on Jun. 18, 2021.

(51) Int. Cl. A61K 35/17; A61K 39/00; A61P 37/04; C07K 16/28; C12N 9/64

(52) U.S. Cl. CPC A61K 35/17; A61K 39/001112; A61K 39/4611; A61K 39/4631; A61K 39/464459; A61K 39/46446; A61P 37/04; C07K 16/2803; C12N 9/6408; C12N 9/641; C12N 9/6491; A61K 2239/13; A61K 2239/21; A61K 2239/22

(57) ABSTRACT

Embodiments relate to a modified cell comprising a polynucleotide encoding an antigen binding molecule and a polynucleotide encoding an agent targeting one or more extracellular matrix (ECM) molecules. In embodiments, the polynucleotide encoding the agent comprises at least a nucleic acid encoding Cathepsin K (CK), a nucleic acid encoding Neutrophil Elastase (NE), or a nucleic acid encoding MMP7. In embodiments, the nucleic acid encoding NE comprises a nucleic acid encoding NE and a nucleic acid encoding a signaling domain of IL2. In embodiments, expression of the polynucleotide encoding the agent is regulated by hypoxia-inducible factor 1-alpha (HIF1α), nuclear factor of activated T-cells (NFAT), forkhead box P3 (FOXP3), or nuclear factor kappa B (NF-κB).

### CROSS REFERENCE TO RELATED APPLICATIONS

This application claims the benefit of U.S. Provisional Application No. 63/218,020, filed on Jul. 2, 2021, and U.S. Provisional Application No. 63/212,303, filed on Jun. 18, 2021, which are incorporated herein by reference in their entirety.

### REFERENCE TO SEQUENCE LISTING

The Sequence Listing associated with this application is provided in text format in lieu of a paper copy and is hereby incorporated by reference into the specification. The name of the text file containing the Sequence Listing is SDS1.0112PCT Final Sequence Listing_ST25.txt. The text file is 207,444 bytes, was created on Jun. 3, 2022, and is being submitted electronically via EFS-Web.

### BACKGROUND

T cells genetically targeted to certain malignancies have demonstrated tremendous clinical outcomes. Physicians draw patients' blood during chimeric antigen receptor (CAR) T cell therapy and harvest their cytotoxic T cells. Subsequently, the cells are re-engineered in a lab to attack the patient's particular cancer. However, recent progress in genome editing technologies allows scientists to disrupt gene expression in T cells to enhance effector functions or overcome tumor immune suppression and metabolically hostile tumor microenvironment.

### SUMMARY

Embodiments relate to a modified cell comprising a polynucleotide encoding an antigen binding molecule and a polynucleotide encoding an agent targeting one or more extracellular matrix (ECM) molecules. In embodiments, the polynucleotide encoding the agent comprises at least a nucleic acid encoding Cathepsin K (CK), a nucleic acid encoding Neutrophil Elastase (NE), or a nucleic acid encoding MMP7. In embodiments, the polynucleotide encoding the agent comprises nucleic acids encoding CK, NE, and MMP7 or comprises two of the nucleic acids encoding CK, NE, and MMP7. In embodiments, the nucleic acid encoding NE comprises a nucleic acid encoding NE and a nucleic acid encoding a signaling peptide of IL2. In embodiments, expression of the polynucleotide encoding of the agent is regulated by hypoxia-inducible factor 1-alpha (HIF1a), nuclear factor of activated T-cells (NFAT), forkhead box P3 (FOXP3), or nuclear factor kappa B (NF-κB).

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 shows exemplary constructs and exemplary modified cells.

FIG. 2 shows the expression of GUC2C CAR and GUCY2C-NFAT-CK+(IL2 SP)NE constructs. CK: Cathepsin K; IL2 SP: IL2 Signal Peptide: and NE: Neutrophil Elastase.

FIG. 3 shows amplification of CAR T cells and mRNA levels of GCC CAR, CK, and NE in the CAR T cells.

FIG. 4 shows cytokine release and proliferation levels of GCC CAR T cells and GCC CAR CK+NE cells after these cells were co-cultured with T84 cells, which are substrate cells of GCC CAR.

FIG. 5 shows mRNA levels of NE and CK in GCC CAR T cells cultured alone and co-cultured with T84, respectively.

FIG. 6 shows NE and CK released by GCC CAR T cells cultured alone and co-cultured with T84.

FIG. 7 shows Western Blot results of ECM molecules degraded by various solutions.

FIG. 8 shows levels of mRNA GCC CAR CK+NE cells in the CoupledCAR® system.

FIG. 9 shows NE and CK released by GCC CAR CK+NE in the CoupledCAR® system.

FIG. 10 shows constructs corresponding to ECM molecules.

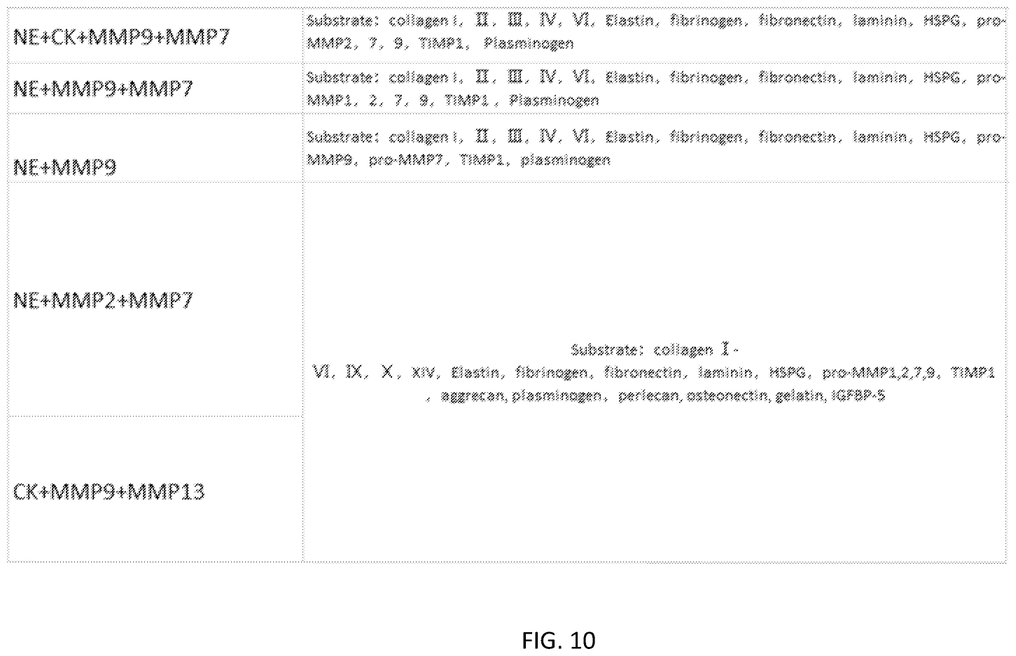

FIG. 11 shows constructs, including functional components and safety components that enable modified cells to degrade ECM molecules while avoiding risks associated with the functional components.

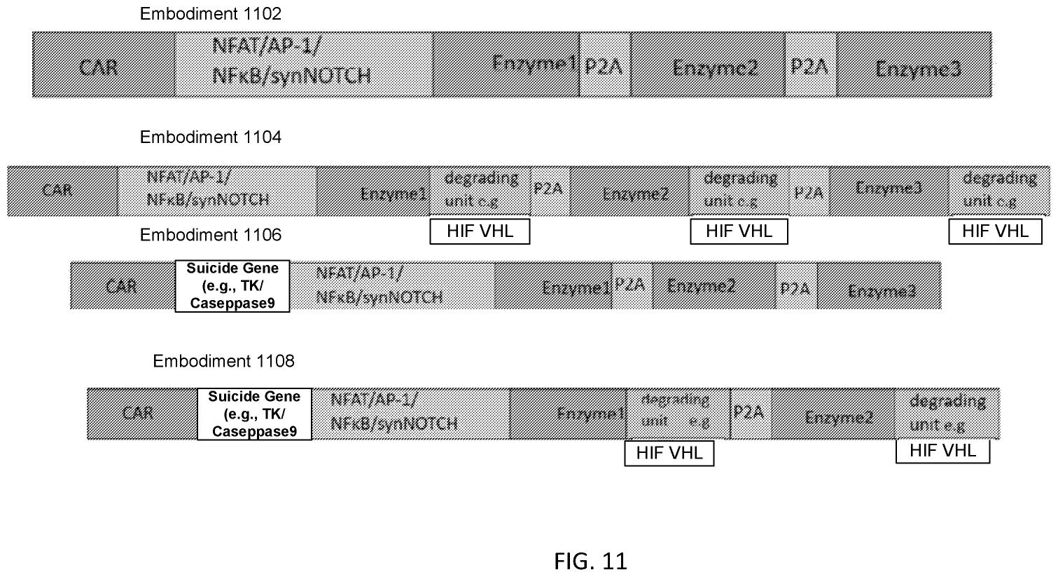

FIG. 12 shows flow cytometry of the expression of CK and CD19 CAR or ACPP CAR on T cells.

FIG. 13 shows mRNA levels of CK in T cells.

FIG. 14 compares NE released by T cells transduced with CARs and NE driven by different SPs.

FIG. 15 shows flow cytometry of MMP7 and CD19 CAR or ACPP CAR expression on T cells.

FIG. 16 shows mRNA levels of CK in T cells.

FIG. 17 shows MMP-7 release in T cells.

FIG. 18 shows flow cytometry of NE, CK, MMP7, and GUCY2C CAR expression on T cells. NT: non-transduced.

FIG. 19 shows flow cytometry of MMP9 and CD19 CAR expression on T cells.

FIG. 20 shows mRNA levels of MMP9 in T cells.

### DETAILED DESCRIPTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight, or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms, including, for example, polyclonal antibodies, monoclonal antibodies, Fv, Fab, and F(ab)₂, as well as single chain antibodies and humanized antibodies (Harlow et al., 1999, In Using Antibodies: A Laboratory Manual, Cold Spring Harbor Laboratory Press, NY; Harlow et al., 1989, In Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragments” refers to a portion of a full length antibody, for example, the antigen binding or variable region of the antibody. Other examples of antibody fragments include Fab, Fab′, F(ab′)2, and Fv fragments; diabodies; linear antibodies; single-chain antibody molecules; and multi-specific antibodies formed from antibody fragments.

The term “Fv” refers to the minimum antibody fragment, which contains a complete antigen-recognition and -binding site. This fragment consists of a dimer of one heavy- and one light-chain variable region domain in a tight, non-covalent association. From the folding of these two domains emanates six hypervariable loops (3 loops each from the H and L chain) that contribute to the amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody that is generated using recombinant DNA technology, such as, for example, an antibody expressed by a bacteriophage. The term also includes an antibody that has been generated by the synthesis of a DNA molecule encoding the antibody and the expression of the DNA molecule to obtain the antibody or to obtain an amino acid encoding the antibody. Synthetic DNA is obtained using technology that is available and well known in the art.

The term “antigen” refers to a molecule that provokes an immune response, which may involve either antibody production, the activation of specific immunologically-competent cells, or both. Antigens include any macromolecule, including all proteins or peptides or molecules derived from recombinant or genomic DNA. For example, DNA includes a nucleotide sequence or a partial nucleotide sequence encoding a protein or peptide that elicits an immune response and, therefore, encodes an “antigen,” as the term is used herein. An antigen need not be encoded solely by a full-length nucleotide sequence of a gene. An antigen can be generated, synthesized, or derived from a biological sample, including a tissue sample, a tumor sample, a cell, or a biological fluid.

The term “anti-tumor effect,” as used herein, refers to a biological effect associated with a decrease in tumor volume, a decrease in the number of tumor cells, a decrease in the number of metastases, a decrease in tumor cell proliferation, a decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies of the prevention the occurrence of tumors in the first place.

The term “auto-antigen” refers to an antigen mistakenly recognized by the immune system as being foreign. Auto-antigens include cellular proteins, phosphoproteins, cellular surface proteins, cellular lipids, nucleic acids, and glycoproteins, including cell surface receptors.

The term “autologous” is used to describe a material derived from a subject that is subsequently re-introduced into the same subject.

The term “allogeneic” is used to describe a graft derived from a different subject of the same species. As an example, a donor subject may be a related or unrelated recipient subject, but the donor subject has immune system markers which are similar to the recipient subject.

The term “xenogeneic” is used to describe a graft derived from a subject of a different species. As an example, the donor subject is from a different species than a recipient subject, and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Throughout this specification, unless the context requires otherwise, the words “comprise,” “includes,” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that no other elements may be present.

The phrase “consisting essentially of” is meant to include any elements listed after the phrase and can include other elements or steps that do not interfere with or contribute to the activity or action specified in the disclosure for the listed elements or steps. Thus, the phrase “consisting essentially of” indicates that the listed elements or steps are required or mandatory, but those other elements are optional and may or may not be present depending upon whether or not they affect the activity or action of the listed elements or steps. In embodiments, those elements or steps that do not affect an embodiment are those elements or steps that do not alter the embodiment's ability in a statistically significant manner to perform a function in vitro or in vivo, such as killing cancer cells in vitro or in vivo.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules, or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein, or (b) a peptide or polypeptide having an amino acid sequence that is substantially identical to a sequence of amino acids in a reference peptide or protein.

The term “co-stimulatory ligand” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor and a ligand that specifically binds with B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, in combination with a primary signal, such as TCR/CD3 ligation, that leads to T cell proliferation and/or upregulation or downregulation of key molecules.

The terms “co-stimulatory signaling region”, “co-stimulatory domain”, and “co-stimulation domain” are used interchangeably to refer to one or more additional stimulatory domain in addition to a stimulatory or signaling domain such as CD3 zeta. The terms “stimulatory” or “signaling” domain (or region) are also used interchangeably, when referring to CD3 zeta. In embodiments, the co-stimulatory signaling domain and the stimulatory signaling domain can be on different molecules in the same cell.

The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated, then the subject's health continues to deteriorate. In contrast, a “disorder” in a subject is a state of health in which the animal is able to maintain homeostasis but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as templates for the synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” refers to the transcription and/or translation of a particular nucleotide sequence driven by its promoter.

The term “expression vector” refers to a vector including a recombinant polynucleotide, including expression control sequences operably linked to a nucleotide sequence to be expressed. An expression vector includes sufficient cis-acting elements for expression; other elements for expression can be supplied by the host cell or in an in a vitro expression system. Expression vectors include all those known in the art, such as cosmids, plasmids (e.g., naked or contained in liposomes), and viruses (e.g., lentiviruses, retroviruses, adenoviruses, and adeno-associated viruses) that incorporate the recombinant polynucleotide.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared to ×100. For example, if 6 of 10 of the positions in two sequences are matched or homologous, then the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins that function as antibodies. The five members included in this class of proteins are IgA, IgG, IgM, IgD, and IgE. IgA is the primary antibody that is present in body secretions, such as saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule, such as a protein or nucleic acid. For example, an “isolated polynucleotide,” as used herein, refers to a polynucleotide that has been purified from the sequences which flank it in a naturally-occurring state, e.g., a DNA fragment that has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment and from association with other components of the cell.

The term “substantially purified” refers to a material that is substantially free from components that are normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In other embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may, in some versions, contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables the integration of genetic information into the host chromosome, resulting in stably transduced genetic information. HIV, SIV, and FIV are all examples of lentiviruses. Vectors derived from lentiviruses offer the means to achieve significant levels of gene transfer in vivo.

The term “modulating” refers to mediating a detectable increase or decrease in the level of a response in a subject compared with the level of a response in the subject in the absence of a treatment or compound and/or compared with the level of a response in an otherwise identical but untreated subject. The term encompasses perturbing and/or affecting a native signal or response, thereby mediating a beneficial therapeutic response in a subject, preferably a human.

Nucleic acid is “operably linked” when it is placed into a functional relationship with another nucleic acid sequence. For example, DNA for a sequence or secretory leader is operably linked to DNA for a polypeptide if it is expressed as a preprotein that participates in the secretion of the polypeptide; a promoter or enhancer is operably linked to a coding sequence if it affects the transcription of the sequence, or a ribosome binding site is operably linked to a coding sequence if it is positioned so as to facilitate translation.

The term “under transcriptional control” refers to a promoter being operably linked to and in the correct location and orientation in relation to a polynucleotide to control the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastases).

Lymphocyte or T cell response in a subject refers to cell-mediated immunity associated with a helper, killer, regulatory, and other types of T cells. For example, T cell response may include activities such as assisting other WBCs in immunologic processes and identifying and destroying virus-infected cells and tumor cells. T cell response in the subject can be measured via various indicators such as a number of virus-infected cells and/or tumor cells that T cells kill, the amount of cytokines (e.g., IL6 and IFNγ) that T cells release in vivo, and/or in co-culturing with virus-infected cells and/or tumor cells, indicates a level of proliferation of T cells in the subject, a phenotype change of T cells, for example, changes to memory T cells, and a level of longevity or lifetime of T cells in the subject.

In embodiments, the method of enhancing the T cell response described herein can effectively treat a subject in need thereof, for example, a subject diagnosed with a tumor, or inhibit the growth of target cells. The term tumor refers to a mass, which can be a collection of fluid, such as blood, or a solid mass. A tumor can be malignant (cancerous) or benign. Examples of blood cancers include chronic lymphocytic leukemia, acute myeloid leukemia, acute lymphoblastic leukemia, and multiple myeloma.

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor antigen | Disease tumor |
|---|---|
| PRLR | Breast Cancer |
| CLCA1 | colorectal cancer |
| MUC12 | colorectal cancer |
| GUCY2C | colorectal cancer and other digestive cancer |
| | types |
| GPR35 | colorectal cancer |
| CR1L | Gastric Cancer |
| MUC 17 | Gastric Cancer |
| TMPRSS11B | esophageal cancer |
| MUC21 | esophageal cancer |
| TMPRSS11E | esophageal cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial cancer |
| UPK2 | Urothelial cancer (including bladder cancer) |
| ADAM12 | Breast cancer, pancreatic cancer, and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL13-Rα2 | Glioma |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinomas |
| EpCAM | Carcinoma |
| EGFRvIII | Glioma-Glioblastoma |
| EGFR | Glioma-NSCL cancer |
| tMUC1 | Cholangiocarcinoma, Pancreatic cancer, |
| | Breast |
| PSCA | pancreas, stomach, or prostate cancer |
| FCER2, GPR18, FCRLA, | breast cancer |
| CXCR5, FCRL3, FCRL2, | |
| HTR3A, and CLEC17A | |
| TRPMI, SLC45A2, and | Lymphoma |
| SLC24A5 | |
| DPEP3 | Melanoma |
| KCNK16 | ovarian, testis |
| LIM2 or KCNV2 | Pancreatic |
| SLC26A4 | thyroid cancer |
| CD171 | Neuroblastoma |
| Glypican-3 | Sarcoma |
| IL13 | Glioma |
| CD79a/b | Lymphoma |
| MAGE A4 | Lung cancer and multiple cancer types |

The term “parenteral administration” of a composition includes, e.g., subcutaneous (s.c.), intravenous (i.v.), intramuscular (i.m.), intrastemal injection, or infusion techniques.

The terms “patient,” “subject,” “individual,” and the like are used interchangeably herein and refer to any human, animal, or living organism amenable to the methods described herein. In certain non-limiting embodiments, the patient, subject, or individual is a human or animal. In embodiments, the term “subject” is intended to include living organisms in which an immune response can be elicited (e.g., mammals). Examples of subjects include humans and animals such as dogs, cats, mice, rats, and transgenic species thereof.

A subject in need of treatment or in need thereof includes a subject having a disease, condition, or disorder that needs to be treated. A subject in need thereof also includes a subject that needs treatment for the prevention of a disease, condition, or disorder.

The term “polynucleotide” or “nucleic acid” refers to mRNA, RNA, CRNA, rRNA, cDNA, or DNA. The term typically refers to a polymeric form of nucleotides of at least 10 bases in length, either ribonucleotides or deoxynucleotides or a modified form of either type of nucleotide. The term includes all forms of nucleic acids, including single and double stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations, inclusive of mutations, additions, deletions, and substitutions, can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogs of the same. Thus, these terms apply to amino acid polymers in which one or more amino acid residues are synthetic non-naturally occurring amino acids, such as a chemical analog of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions, and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “promoter” refers to a DNA sequence recognized by the synthetic machinery of the cell or introduced synthetic machinery required to initiate the specific transcription of a polynucleotide sequence. The term “expression control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” refers to a molecule recognizing and adhering to a particular second molecule in a sample or organism but does not substantially recognize or adhere to other structurally unrelated molecules in the sample. The term “specifically binds,” as used herein with respect to an antibody, refers to an antibody that recognizes a specific antigen but does not substantially recognize or bind other molecules in a sample. For example, an antibody that specifically binds an antigen from one species may also bind that antigen from another species, but such cross-species reactivity does not itself alter the classification of an antibody as specific. In another example, an antibody that specifically binds an antigen may also bind different allelic forms of the antigen. However, such cross reactivity does not itself alter the classification of an antibody as specific. In some instances, the terms “specific binding” or “specifically binding” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody will reduce the amount of labeled A bound to the antibody.

A “binding protein” is a protein that is able to bind non-covalently to another molecule. A binding protein can bind to, for example, a DNA molecule (a DNA-binding protein), an RNA molecule (an RNA-binding protein), and/or a protein molecule (a protein-binding protein). In the case of a protein-binding protein, it can bind to itself (to form homodimers, homotrimers, etc.), and/or it can bind to one or more molecules of a different protein or proteins. A binding protein can have more than one type of binding activity. For example, zinc finger proteins have DNA-binding, RNA-binding, and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

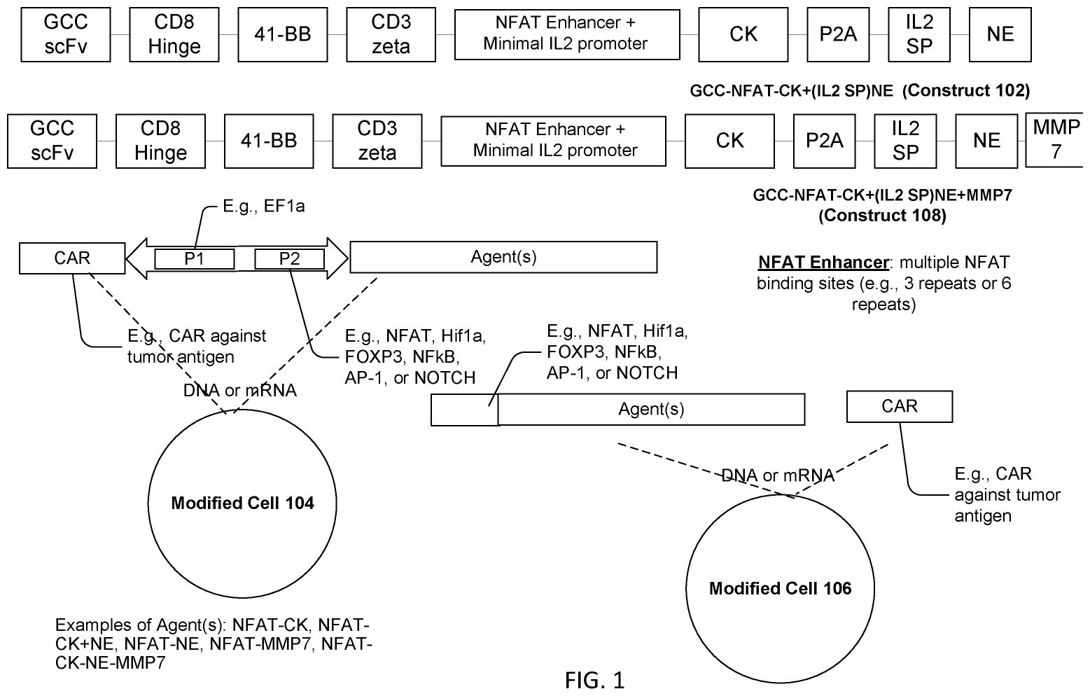

Zinc finger binding domains can be “engineered” to bind to a predetermined nucleotide sequence, for example, via engineering (altering one or more amino acids) of the recognition helix region of a naturally occurring zinc finger protein. Further, a Zinc finger binding domain may be fused with a DNA-cleavage domain to form a Zinc finger nuclease (ZFN) targeting a specific desired DNA sequence. For example, a pair of ZFNs (e.g., a ZFN-left arm and a ZFN-right arm) may be engineered to target and cause modifications of specific desired DNA sequences (e.g., TRAC genes), as illustrated in FIG. 1.

“Cleavage” refers to the breakage of the covalent backbone of a DNA molecule. Cleavage can be initiated by a variety of methods, including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “target site” or “target sequence” is a nucleic acid sequence that defines a portion of a nucleic acid to which a binding molecule will bind, provided sufficient conditions for binding exist. For example, the sequence 5′ GAATTC 3′ is a target site for the Eco RI restriction endonuclease.

A “fusion” molecule is a molecule in which two or more subunit molecules are linked, preferably covalently. The subunit molecules can be the same chemical type of molecule or can be different chemical types of molecules. Examples of the first type of fusion molecule include, but are not limited to, fusion proteins (for example, a fusion between a ZFP DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide and a fusion between a minor groove binder and a nucleic acid.

Expression of a fusion protein in a cell can result from the delivery of the fusion protein to the cell or by delivery of a polynucleotide encoding the fusion protein to a cell, wherein the polynucleotide is transcribed and the transcript is translated to generate the fusion protein. Trans-splicing, polypeptide cleavage, and polypeptide ligation can also be involved in the expression of the protein in a cell. Methods for polynucleotide and polypeptide delivery to cells are presented elsewhere in this disclosure.

“Modulation” of gene expression refers to a change in the activity of a gene. Modulation of expression can include but is not limited to gene activation and gene repression. Genome editing (e.g., cleavage, alteration, inactivation, random mutation) can be used to modulate expression. Gene inactivation refers to any reduction in gene expression as compared to a cell that does not include a ZFP, as described herein. Thus, gene inactivation may be partial or complete.

A “region of interest” is any region of cellular chromatin, such as, for example, a gene or a non-coding sequence within or adjacent to a gene in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences, or introns, or within non-transcribed regions, either upstream or downstream of the coding region. A region of interest can be as small as a single nucleotide pair or up to 2,000 nucleotide pairs in length or any integral value of nucleotide pairs.

By “statistically significant,” it is meant that the result was unlikely to have occurred by chance. Statistical significance can be determined by any method known in art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount and may include a decrease that is about 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein.

The term “stimulation” refers to a primary response induced by binding of a stimulatory molecule (e.g., a TCR/CD3 complex) with its cognate ligand, thereby mediating a signal transduction event, such as signal transduction via the TCR/CD3 complex. Stimulation can mediate altered expression of certain molecules, such as downregulation of TGF-β and/or reorganization of cytoskeletal structures. CD3 zeta is not the only suitable primary signaling domain for a CAR construct with respect to the primary response. For example, back in 1993, both CD3 zeta and FcR gamma were shown as functional primary signaling domains of CAR molecules. Eshhar et al., “Specific activation and targeting of cytotoxic lymphocytes through chimeric single chains consisting of antibody-binding domains and the gamma or zeta subunits of the immunoglobulin and T-cell receptors” PNAS, 1993 Jan. 15; 90(2):720-4, showed that two CAR constructs in which an scFv was fused to “either the FcR y chain or the CD3 complex s chain” triggered T cell activation and the target cell. Notably, as demonstrated in Eshhar et al., CAR constructs containing only the primary signaling domain CD3 zeta or FcR gamma are functional without the co-presence of co-stimulatory domains. Additional non-CD3 zeta based CAR constructs have been developed over the years. For example, Wang et al., “A Chimeric Antigen Receptor (CARs) Based Upon a Killer Immunoglobulin-Like Receptor (KIR) Trigger Robust Cytotoxic Activity in Solid Tumors” Molecular Therapy, vol. 22, no. Suppl. 1, May 2014, page S57, tested a CAR molecule in which an scFv was fused to “the transmembrane and the cytoplasmic domain of” a killer immunoglobulin-like receptor (KIR). Wang et al. state that “a KIR-based CAR targeting mesothelin (SS 1-KIR) triggers antigen-specific cytotoxic activity and cytokine production that is comparable to CD3˜-based CARs.” A second publication from the same group, Wang et al., “Generation of Potent T-cell Immunotherapy for Cancer Using DAP12-Based, Multichain, Chimeric Immunoreceptors” Cancer Immunol Res. 2015 July; 3(7):815-26, showed that a CAR molecule in which “a single-chain variable fragment for antigen recognition [was fused] to the transmembrane and cytoplasmic domains of KIR2DS2, a stimulatory killer immunoglobulin-like receptor (KIR)” functioned both in vitro and in vivo “when introduced into human T cells with DAP12, an immunotyrosine-based activation motifs-containing adaptor.”

The term “stimulatory molecule” refers to a molecule on a T cell that specifically binds a cognate stimulatory ligand present on an antigen presenting cell. For example, a functional signaling domain derived from a stimulatory molecule is the zeta chain associated with the T cell receptor complex.

The term “stimulatory ligand” refers to a ligand that, when present on an antigen presenting cell (e.g., an APC, a dendritic cell, a B-cell, and the like.), can specifically bind with a cognate binding partner (referred to herein as a “stimulatory molecule”) on a cell, for example, a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to the treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor, or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease, its severity, and the age, weight, etc., of the subject to be treated.

The term “treat a disease” refers to the reduction of the frequency or severity of at least one sign or symptom of a disease or disorder experienced by a subject.

The term “transfected,” “transformed,” or “transduced” refers to a process by which an exogenous nucleic acid is transferred or introduced into the host cell. A “transfected” or “transformed” or “transduced” cell is one that has been transfected, transformed, or transduced with the exogenous nucleic acid. The cell includes the primary subject cell and its progeny.

The term “vector” refers to a polynucleotide that comprises an isolated nucleic acid and which can be used to deliver the isolated nucleic acid to the interior of a cell. Numerous vectors are known in the art, including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds that facilitate the transfer of nucleic acid into cells, such as polylysine compounds, liposomes, and the like. Examples of viral vectors include adenoviral vectors, adeno-associated virus vectors, retroviral vectors, and others. For example, lentiviruses are complex retroviruses, which, in addition to the common retroviral genes gag, pol, and env, contain other genes with regulatory or structural functions. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes; for example, the genes env, vif, vpr, vpu, and nef are deleted, making the vector biologically safe.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

T cells, or T lymphocytes, are a type of white blood cell of the immune system. There are various types of T cells including T helper (T_{H}) cells, cytotoxic T (T_{C}) cells (T killer cells, killer T cells), natural killer T (NKT) cells, memory T (Tm) cells, regulatory T (Treg) cells, and gamma delta T (γδ T) cells.

T helper (T_{H}) cells assist other lymphocytes, for example, activating cytotoxic T cells and macrophages and maturation of B cells into plasma cells and memory B cells. These T helper cells express CD4 glycoprotein on their surface and are also known as CD4⁺ T cells. Once activated, these T cells divide rapidly and secrete cytokines.

Cytotoxic T (T_{C}) cells destroy virus-infected cells and tumor cells and are also involved in transplant rejection. They express CD8 protein on their surface. Cytotoxic T cell release cytokines.

Natural Killer T (NKT) cells are different from natural killer cells. NKT cells recognize glycolipid antigens presented by CD1d. Once activated, NKT cells produce cytokine and release cell killing molecules.

Memory T (Tm) cells are long-lived and can expand to large number of effector T cells upon re-exposure to their cognate antigen. Tm cells provide the immune system with memory against previously encountered pathogens. There are various subtypes of Tm cells including central memory T (T_{CM}) cells, effector memory T (T_{EM}) cells, tissue resident memory T (T_{RM}) cells, and virtual memory T cells. Tm cells are either CD4⁺ or CD8⁺ and usually CD45RO.

Regulatory T (Treg) cells shut down T cell mediated immunity at the end of an immune reaction and suppress autoreactive T cells that escaped the process of negative selection in the thymus. Subsets of Treg cells include thymic Treg and peripherally derived Treg. Both subsets of Treg require the expression of the transcription factor FOXP3.

Gamma delta T (γδ T) cells are a subset of T cells that possess a γδ T cell receptor (TCR) on the cell surface, as most T cells express the αβ TCR chains. γδ T cells are less common in human and mice and are mainly found in the gut mucosa, skin, lung, and uterus. They are involved in the initiation and propagation of immune responses.

Tumor ECM is a matrix composed of multiple components wrapped around a solid tumor and plays a vital role in tumor diseases. Tumor ECM has many functions: for example, providing support for tumor cells, fixing and protecting barriers, regulating the dynamic behavior of tumor cells, and providing necessary signals for tumor cell growth.

Embodiments of the present disclosure enable CAR T cells to secrete enzymes that can degrade components of ECM. Embodiments of the present disclosure further provide a mechanism to regulate the expression of these enzymes. Examples of such enzymes include MMP1, MMP2, MMP3, MMP7, MMP8, MMP9, MMP13, cathepsin L, cathepsin K, neutrophil elastase, plasmin, and trypsin. With ECM degraded, CAR T cells may infiltrate. For example, in colorectal cancer tissues, the highest expressed ECM components are collagen VI, heparan sulfate, fibrillin, fibronectin, collagen I, collagen IV, and collagen III. Collagen I, collagen IV, and fibronectin are the main components, which are cross-linked to form a network structure. Thus, the degradation of collagen I, collagen IV, and fibronectin can destroy the ECM and, thereby, reshaping the ECM, and exposing tumor cells to CAR T cells.

Embodiments describe a polynucleotide encoding one or more agents, and the one or more agents comprising at least one of cathepsin K (CK), neutrophil elastase (NE), and matrix metalloproteinase-7 (MMP7). Embodiments further describe a vector, a composition, and/or a cell comprising the polynucleotide encoding the one or more agents.

Embodiments describe a polynucleotide comprising a nucleic acid encoding a CAR and a nucleic acid encoding the one or more agents, and the one or more agents comprising at least one of cathepsin K (CK), neutrophil elastase (NE), and matrix metalloproteinase-7 (MMP7). Embodiments further describe a vector, a composition, and/or a cell comprising the polynucleotide encoding one or more of these agents.

Embodiments describe a modified cell engineered to express and secrete one or more agents targeting one or more extracellular matrix (ECM) molecules, the modified cell comprising a polynucleotide encoding a CAR and a polynucleotide encoding one or more agents, and the one or more agents comprising at least one of cathepsin K (CK), neutrophil elastase (NE), and matrix metalloproteinase-7 (MMP7).

Embodiments describe a method of inducing or causing a T cell response, the method comprising: contacting a cell comprising a tumor antigen with a modified cell comprising a CAR, wherein the CAR binds the tumor antigen, and the T cell response comprises IFNγ release.

In embodiments, the polynucleotide encoding the one or more agents comprises a nucleic acid comprising an NFAT binding site, a nucleic acid encoding the one or more agents, and a nucleic acid encoding an oxygen-dependent degradation domain of HIF1α, wherein the nucleic acid encoding the agent is flanked by the NFAT binding site and the nucleic acid encoding the oxygen-dependent degradation domain of HIF1α.

Von Hippel-Lindau tumor-suppressor protein (VHL) interacts with or binds the oxygen-dependent degradation (ODD) domain of HIF1α. The ODD domain of HIF1a is located in its central region and consists of 200 amino acid residues. The ODD domain controls the degradation of HIF1a by the ubiquitin-proteasome pathway, and the deletion of this entire region is required for DNA binding and transactivation in the absence of hypoxic signaling. The product of VHL gene was reported to mediate ubiquitination and proteasomal degradation of HIF1a under normoxic conditions by interaction with the core of the ODD domain of HIF1α.

In embodiments, the expression of the one or more agents is regulated by HIF1α, NFAT, FOXP3, or NF-κB.

In embodiments, the one or more agents comprise CK. In embodiments, the one or more agents comprise CK and NE, CK and MMP7, or CK, NE, and MMP7. In embodiments, the polynucleotide encoding the one or more agents comprises: (1) SEQ ID NOs: 5 and 6; and (2) a nucleotide encoding SEQ ID NO: 7. In embodiments, the polynucleotide encoding the one or more agents comprises: (1) SEQ ID NO: 48, 52, or 54; or (2) a nucleic acid encoding SEQ ID NO: 51 or 53.

In embodiments, the one or more agents comprise NE. In embodiments, the polynucleotide encoding the one or more agents comprises a nucleic acid encoding a signaling peptide of IL2 and a nucleic acid encoding NE. In embodiments, the polynucleotide encoding the one or more agents comprises SEQ ID NO: 10 and a nucleic acid encoding SEQ ID NO: 42 or 43; SEQ ID NO: 11 or 12; or SEQ ID NO: 9 and a nucleic acid encoding SEQ ID NO: 10.

In embodiments, the one or more agents comprise MMP7. In embodiments, the one or more agents comprise CK and NE, CK and MMP7, or CK, NE, and MMP7. In embodiments, the polynucleotide encoding the one or more agents comprises: (1) SEQ ID NO: 52 or 54; or (2) a nucleic acid encoding SEQ ID NO: 51 or 53. In embodiments, the one or more agents comprise CK, NE, and MMP7. In embodiments, the one or more agents comprise NE and MMP. In embodiments, the polynucleotide encoding the one or more agents comprises: (1) SEQ ID NO: 50 or 54; or (2) a nucleic acid encoding SEQ ID NO: 49.

In embodiments, one or more ECM molecules comprise at least one of collagen I, collagen III, collagen VI, collagen IV, and fibronectin.

In embodiments, the modified cell comprises a dominant negative form of an inhibitory immune checkpoint molecule or a receptor thereof, and the inhibitory immune checkpoint molecule is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig, and ITIM domain (TIGIT), leukocyte-associated immunoglobulin-like receptor 1 (LAIRI), natural killer cell receptor 2B4 (2B4), and CD 160. In embodiments, the modified cell has a reduced expression of the endogenous TRAC gene.

The tumor microenvironment (TME) includes tumor cells, vasculature, ECM, stromal, and immune cells. The ECM includes numerous molecules (ECM molecules) classified traditionally into collagens, elastin, and microfibrillar proteins, proteoglycans including hyaluronan, and noncollagenous glycoproteins. The tumor ECM or extra-tumor matrix facilitates the growth of the tumor and resistance to therapies. A summary of ECM can be found at Järveläinen H, Sainio A, Koulu M, Wight T N, Penttinen R. ECM molecules: potential targets in pharmacotherapy. Pharmacol Rev. 2009; 61(2): 198-223. doi:10.1124/pr. 109.001289, which is incorporated herein in its entirety. In embodiments, examples of the ECM molecules include collagen I, collagen III, collagen VI, collagen IV, and fibronectin. In embodiments, the agent targeting the ECM molecules refers to an agent that degrades and/or causes, induces, or increases the degradation of one or more ECM molecules including ECM enzymes. In embodiments, the agent targeting the ECM includes an agent targeting the synthesis of the ECM, including cytokine inhibitors and cytokines. Examples of an agent targeting the synthesis of the ECM include antibodies to TGF-β1, antibodies to TGF-β2, TGF-β1 signaling inhibitors, TGF-β receptor I kinase inhibitors, antibodies to CTGF, recombinant TGF-β3, and/or recombinant interleukin-10.

Embodiments describe a polynucleotide encoding an agent targeting ECM. Embodiments further describe a vector, a composition, and/or a cell comprising the polynucleotide encoding the agent. In embodiments, the agent targeting the ECM includes an agent targeting the degradation of the ECM. Examples of the agent targeting the degradation of the ECM include MMP inhibitors, collagenase, and MMP-14 inhibitors, broad-spectrum MMP inhibitors, selective cathepsin K inhibitors, heparanase activity inhibitors, and/or collagenase stimulators. In embodiments, the agent targeting the ECM includes an agent targeting the signaling of the ECM. Examples of the agent targeting the signaling of the ECM include antibodies to αv/β3 integrin, antibodies to α4/β7 integrin, and/or antibodies to α5/β1 integrin. In embodiments, the agent targeting the ECM includes an agent that degrades one or more ECM molecules, such as collagen I, collagen III, collagen VI, collagen IV, and fibronectin. In embodiments, the agent targeting the ECM includes one or more enzymes that degrade the ECM molecules. Examples of agents that degrade one or more ECM molecules include MMP1, MMP2, MMP3, MMP7, MMP8, MMP9, MMP13, cathepsin L (CL), cathepsin K (CK), neutrophil elastase (NE), plasmin, and trypsin. In embodiments, CAR T cells may secrete enzymes that can degrade the ECM molecules at the tumor site to break TME. Thus, CAR T cells can be made more accessible to more tumor cells. In addition, once the barrier is broken, the growth of tumor cells lacking the necessary signals slows down, and thus CAR T cells are more likely to infiltrate the solid tumor.

In embodiments, the compositions and/or methods described herein can be combined with techniques associated with CoupledCAR® described in PCT Publication Nos: WO2020106843 and WO2020146743, which are incorporated by reference herein in their entirety.

Embodiments describe a polynucleotide comprising a polynucleotide encoding an antigen binding molecule and a polynucleotide encoding an agent targeting an extracellular matrix (ECM) molecule.

Embodiments describe a composition comprising a polynucleotide encoding an antigen binding molecule and a polynucleotide encoding an agent targeting an ECM molecule.

Embodiments describe a modified cell engineered to express an antigen binding molecule and one or more ECM molecules.

Embodiments describe a vector comprising the polynucleotide. Embodiments describe a cell comprising the vector. Embodiments describe a method of enhancing the treatment of cancer, the method comprising: administering a pharmaceutical composition comprising a viral vector comprising the polynucleotide.

Embodiments describe a modified cell engineered to express an antigen binding molecule and one or more agents targeting an ECM molecule, wherein the expression and/or function of an agent targeting one or more ECM molecules in the modified cell has been enhanced. Modified cells described herein include T cells, NK cells, dendritic cells, or a macrophages.

Modified T cells can be derived from a stem cell. The stem cells may be adult stem cells, embryonic stem cells, more particularly non-human stem cells, cord blood stem cells, progenitor cells, bone marrow stem cells, induced pluripotent stem cells, totipotent stem cells, or hematopoietic stem cells. A modified cell may also be a dendritic cell, an NK-cell, a B-cell, or a T-cell selected from the group consisting of inflammatory T-lymphocytes, cytotoxic T-lymphocytes, regulatory T-lymphocytes, or helper T-lymphocytes. In another embodiment, Modified cells may be derived from the group consisting of CD4+T-lymphocytes and CD8+T-lymphocytes. Prior to expansion and genetic modification of the cells of the invention, a source of cells may be obtained from a subject through a variety of non-limiting methods. T cells may be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art may be used. In embodiments, modified cells may be derived from a healthy donor, from a patient diagnosed with cancer, or from a patient diagnosed with an infection. In embodiments, the modified cell is part of a mixed population of cells that present different phenotypic characteristics.

The term “stem cell” refers to any of certain types of cells which have the capacity for self-renewal and the ability to differentiate into other kinds of a cell. For example, a stem cell gives rise either to two daughter stem cells (as occurs in vitro with embryonic stem cells in culture) or to one stem cell and a cell that undergoes differentiation (as occurs, e.g., in hematopoietic stem cells, which give rise to blood cells). Different categories of stem cells may be distinguished on the basis of their origin and/or on the extent of their capacity for differentiation into other types of cells. For example, the stem cell may include embryonic stem (ES) cells (i.e., pluripotent stem cells), somatic stem cells, Induced pluripotent stem cells, and any other types of stem cells.

The pluripotent embryonic stem cells may be found in the inner cell mass of a blastocyst and have a high innate capacity for differentiation. For example, pluripotent embryonic stem cells may have the potential to form any type of cell in the body. When grown in vitro for long periods of time, ES cells maintain pluripotency: progeny cells retain the potential for multilineage differentiation.

Somatic stem cells may include the fetal stem cells (from the fetus) and adult stem cells (found in various tissues, such as bone marrow). These cells have been regarded as having a capacity for differentiation lower than that of the pluripotent ES cells—with the capacity of fetal stem cells being greater than that of adult stem cells; they apparently differentiate into only a limited range of types of cell and have been described as multipotent. The ‘tissue-specific’ stem cells normally give rise to only one type of cell. For example, embryonic stem cells may be differentiated into blood stem cells (e.g., hematopoietic stem cells (HSCs)), which may be further differentiated into various blood cells (e.g., red blood cells, platelets, white blood cells, etc.).

Induced pluripotent stem cells (i.e., iPS cells or iPSCs) may include a type of pluripotent stem cell artificially derived from a non-pluripotent cell (e.g., an adult somatic cell) by inducing the expression of specific genes. Induced pluripotent stem cells are similar to natural pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells may be made from the adult stomach, liver, skin cells, and blood cells.

Embodiments describe a method of stimulating T cell response and treating a subject having a form of cancer or enhancing T cell response and/or treatment of cancer. The method comprises administering an effective amount of a modified cell and administering an effective amount of an agent targeting one or more ECM molecules. In embodiments, the modified cells and the agent may be administered separately, sequentially, and/or simultaneously.

Embodiments describe a method of enhancing T cell response (IL6, TNFα, and/or IFNγ release and/or cell expansion), the method comprising administering an effective amount of a modified cell comprising a polynucleotide encoding an antigen binding molecule and a polynucleotide encoding an agent targeting one or more ECM molecules, and the T cell response is enhanced as compared with administering an effective amount of the modified cell comprising the polynucleotide encoding the antigen binding molecule without the polynucleotide encoding an agent targeting one or more ECM molecules.

In embodiments, the expression of the agent targeting one or more molecules ECM is regulated by a promoter of HIF1α, NFAT, FOXP3, or NFkB.

In embodiments, the polynucleotide encoding the agent targeting one or more ECM molecules comprises a polynucleotide encoding cathepsin K (CK).

In embodiments, the polynucleotide encoding the agent targeting one or more ECM molecules comprises a polynucleotide encoding CK and/or a polynucleotide encoding NE.

In embodiments, the polynucleotide encoding NE comprises a polynucleotide encoding NE and a polynucleotide encoding a signaling domain of IL2 (e.g., SEQ ID NO: 42 or 43).

In embodiments, the polynucleotide encoding the agent targeting one or more ECM molecules comprises a polynucleotide encoding CK, a polynucleotide encoding NE, and a polynucleotide encoding MMP7.

In embodiments, the polynucleotide comprises (i) at least one of SEQ ID NOs: 5-10 and 16 and (ii) at least one of SEQ ID NOs: 11-47. In embodiments, the polynucleotide comprises (i) at least one of SEQ ID NOs: 5-10, (ii) SEQ ID NO: 13, and (iii) SEQ ID NO: 11 or 12.

In embodiments, the viral vector comprises a lentivirus. In embodiments, the viral vector comprises an rAAV particle comprising an AAV capsid protein and a vector comprising a nucleic acid encoding an antigen binding molecule inserted between a pair of AAV inverted terminal repeats (ITRs) in a manner effective for infecting cells, organs, or tissues of the peripheral system in a subject such that the cells, organs or tissues express the antigen binding molecule.

In embodiments, the modified cell comprises a polynucleotide encoding an antigen binding molecule and a polynucleotide encoding an agent targeting one or more ECM molecules.

In embodiments, the one or more ECM molecules comprise collagen I, collagen III, collagen VI, collagen IV, and/or fibronectin.

In embodiments, the agent targeting the ECM includes an agent that degrades one or more ECM molecules

In embodiments, the agent targeting one or more ECM molecules comprises MMP1, MMP2, MMP3, MMP7, MMP8, MMP9, MMP13, cathepsin L (CL), cathepsin K (CK), neutrophil elastase (NE), plasmin, and/or trypsin.

In embodiments, the agent targeting one or more ECM molecules comprises MMP7, NE, and/or CK.

In embodiments, the antigen binding molecule is a CAR, which comprises an antigen-binding domain, a transmembrane domain, and an intracellular signaling domain.

In embodiments, the antigen-binding domain binds to a tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL13Ra2, Mesothelin, IL11Ra, PSCA, PRSS21, VEGFR2, Lewis Y, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In embodiments, the intracellular signaling domain comprises a co-stimulatory signaling domain, or a primary signaling domain and a co-stimulatory signaling domain, wherein the co-stimulatory signaling domain comprises a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In embodiments, the antigen binding molecule is a modified TCR.

In embodiments, the TCR is derived from spontaneously occurring tumor-specific T cells in patients. In embodiments, the TCR binds to a tumor antigen. In embodiments, the tumor antigen comprises CEA, gp100, MART-1, p53, MAGE-A3, or NY-ESO-1. In embodiments, the TCR comprises TCRγ and TCRδ Chains or TCRα and TCRβ chains, or a combination thereof.

In embodiments, the cell is an immune effector cell (e.g., a population of immune effector cells). In embodiments, the immune effector cell is a T cell or an NK cell. In embodiments, the immune effector cell is a T cell. In embodiments, the T cell is a CD4+ T cell, a CD8+ T cell, or a combination thereof. In embodiments, the cell is a human cell.

In embodiments, the enhanced expression and/or function of the agent targeting one or more ECM molecules is implemented by introducing a nucleic acid of the one or more genes, which is present in the modified cell in a recombinant DNA construct, in an mRNA, or in a viral vector. In embodiments, the one or more genes are related to biosynthesis or transportation pathway of the one or more ECM molecules. In embodiments, the nucleic acid is an mRNA, which is not integrated into the genome of the modified cell.

In embodiments, the nucleic acid is associated with an oxygen-dependent domain. In embodiments, the oxygen-dependent degradation domain comprises a HIF1α domain that binds VHL. In embodiments, the nucleic acid is regulated by a promoter comprising a binding site for a transcription modulator that modulates the expression and/or secretion of one or more therapeutic agents in the cell. In embodiments, the transcription modulator is or includes HIF1α, NFAT, FOXP3, and/or NFkB. In embodiments, the one or more therapeutic agents include one or more cytokines such as IL6, IL12, and IFNγ, and/or one or more ECM molecules such as one or more ECM enzymes

Embodiments describe a composition comprising a first population of cells comprising a first CAR binding a first antigen and a second population of cells comprising a second CAR binding a second antigen, wherein the second antigen is a tumor antigen and is different from the first antigen, and the first population and/or the second population of cells comprise one or more polynucleotides, as described above.

Embodiments describe the use of the composition or a method of enhancing the expansion of cells in a subject in need thereof or treating a subject having cancer, the method comprising: administering an effective amount of the pharmaceutical composition described herein to the subject, the subject having a form of cancer expressing a tumor antigen.

In embodiments, expansion of the second population of cells in the subject is greater than the expansion of the second population of cells in a subject that is administered with the second population of cells but not the first population of cells. In embodiments, the expansion is measured based on numbers of the second population of cells or copy numbers of DNA encoding the second CAR. In embodiments, the cells are T cells, NK cells, macrophages, or dendritic cells.

In embodiments, the first antigen comprises a cell surface molecule of a white blood cell (WBC), a tumor antigen, or a solid tumor antigen. In embodiments, the WBC is a granulocyte, a monocyte, or a lymphocyte. In embodiments, the WBC is a B cell.

In embodiments, the cell surface molecule of the WBC is CD19, CD22, CD20, BCMA, CD5, CD7, CD2, CD16, CD56, CD30, CD14, CD68, CD11b, CD18, CD169, CD1c, CD33, CD38, CD138, or CD13. In embodiments, the cell surface molecule of the WBC is CD19, CD20, CD22, or BCMA. In embodiments, the cell surface molecule of the WBC is CD19 or BCMA.

In embodiments, the tumor antigen is a solid tumor antigen.

Embodiments describe a method of enhancing NE expression by CAR cells, the method comprising: introducing a polynucleotide encoding a CAR into a cell to obtain a CAR cell; and introducing a polynucleotide encoding NE and an SP of IL2, wherein the expression and release of NE by the cell is enhanced as compared to a CAR cell having the polynucleotide encoding the agent without the SP of IL2. In embodiments, the cell is NK or T cell. In embodiments, the polynucleotide comprises the polynucleotide sequence SEQ ID NOs: 5 and 6 and a polynucleotide encoding the amino acid sequences SEQ ID NOs: 7, 9, 10, and 16.

In embodiments, the polynucleotide comprises the polynucleotide sequence SEQ ID NOs: 5 and 6 and a polynucleotide encoding the amino acid sequences SEQ ID NOs: 7 and 9.

In embodiments, the polynucleotide comprises the polynucleotide sequence SEQ ID NOs: 5 and 6 and a polynucleotide encoding the amino acid sequences SEQ ID NOs: 9 and 10.

In embodiments, the polynucleotide comprises the polynucleotide sequence SEQ ID NOs: 5 and 6 and a polynucleotide encoding the amino acid sequences SEQ ID NOs: 9 and 16.

In embodiments, the polynucleotide comprises the polynucleotide sequence SEQ ID NOs: 5 and 6 and a polynucleotide encoding the amino acid sequences SEQ ID NOs: 7, 9, and 10.

In embodiments, the polynucleotide comprises the polynucleotide sequence SEQ ID NOs: 5 and 6 and a polynucleotide encoding the amino acid sequences SEQ ID NOs: 9 and 16.

In embodiments, the polynucleotide comprises the polynucleotide sequence SEQ ID NOs: 5 and 6 and a polynucleotide encoding the amino acid sequences SEQ ID NOs: 9, 10, and 16.

In embodiments, the modified cells comprise a nucleic acid encoding a binding molecule and a dominant negative form of an inhibitory immune checkpoint molecule or a receptor thereof. In embodiments, the inhibitory immune checkpoint molecule is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD 160. In embodiments, the inhibitory immune checkpoint molecule is modified PD-1.

In embodiments, the modified PD-1 lacks a functional PD-1 intracellular domain for PD-1 signal transduction, interferes with a pathway between PD-1 of a human T cell of the human cells and PD-L1 of a certain cell, comprises or is a PD-1 extracellular domain or a PD-1 transmembrane domain, comprises a modified PD-1 intracellular domain comprising a substitution or deletion as compared to a wild-type PD-1 intracellular domain, and/or comprises or is a soluble receptor comprising a PD-1 extracellular domain that binds to PD-L1 of a certain cell.

In embodiments, the modified cell has a reduced expression of the endogenous TRAC gene.

In embodiments, the modified cells include a nucleic acid encoding hTERT or a nucleic acid encoding SV40LT, or a combination thereof. In embodiments, the modified cells include a nucleic acid encoding hTERT and a nucleic acid encoding SV40LT. In embodiments, the expression of hTERT is regulated by an inducible expression system. In embodiments, the expression of SV40LT gene is regulated by an inducible expression system. In embodiments, the inducible expression system is rTTA-TRE, which increases or activates the expression of SV40LT gene or hTERT gene, or a combination thereof.

In embodiments, the modified cells described herein further include a nucleic acid encoding a suicide gene. In embodiments, the suicide gene includes an HSV-TK suicide gene system, and/or the modified cell can be induced to undergo apoptosis.

Embodiments relate to a modified cell engineered to express an antigen binding molecule, wherein the expression and/or function of one or more molecules in the modified cell has been reduced or eliminated, the one or more molecules being associated with apoptosis of the modified cell. In embodiments, one or more molecules include glucocorticoid receptor α (GRα), Fas, TNFR1, TNFR2, and/or TRAIL. In embodiments, the modified cell comprises a disruption in an endogenous gene or addition of an exogenous gene that is associated with a biosynthesis or transportation pathway of the one or more molecules.

In embodiments, the amplification of T cells is enhanced by inhibiting the apoptosis of T cells primarily through the FAS, TNFR1/2, and TRAIL signaling pathways. Fas is a transmembrane protein belonging to the tumor necrosis factor receptor superfamily. The binding of Fas to Fas ligand (FasL) can initiate apoptosis signaling. The initiation involves a series of steps: first, the Fas ligand induces receptor trimerization and then forms an apoptosis-inducing complex on the cell membrane, which includes the Fas-associated protein FADD with a death domain. The complex then polymerizes multiple caspase 8, which in turn causes a subsequent cascade reaction, namely the caspase cascade, which activates the caspase zymogens and a cascade of subsequent cellular processes including the cells undergoing apoptosis. The TNF-mediated apoptotic pathway is similar to FAS, which is mediated by the death receptor TNFRI. Tumor necrosis factor receptor (TNFR) is the representative family of the largest death receptors, including TNFRI (p55, CD120a), TNFRII (p75, CD120b), and the like. Their common feature is an intracellular region having a highly homologous amino acid sequence necessary for transducing cell death signals, namely the death domain (DD). Death domain proteins found mainly include FADD, TNFRI associated death domain protein (TRADD), and receptor interacting protein (RIP). TNF is mainly produced by infected and activated macrophages and T cells and mediates apoptosis through its cell surface receptors TNFRI and TNFRII. In embodiments, an apoptotic signal is a tumor necrosis factor related to apoptosis-induced ligand (TRAIL), which is a member of the tumor necrosis factor family and is capable of inducing apoptosis in most human tumor cells. There is no obvious cytotoxicity to normal cells. TRAIL induces tumor cell apoptosis mainly by activating exogenous pathways, accompanied by cascade amplification of endogenous pathways. After TRAIL binds to the death receptor on the cell membrane surface, the intramembranous segment of the death receptor activates and undergoes self-shearing to form death-inducing signaling complexes (DISCs). The recruitment of Fas-associated death domain (FADD) and caspase-8, caspase-10 precursors, is a prerequisite for the formation of DISCs. Caspase-8 and caspase-10 precursors form active cleaved caspase-8 and cleaved caspase-10, which in turn activates caspase-3, 6, and 7 and initiates apoptosis. In embodiments, dominant negative forms of FAS/TNFR1/2 and TRAIL or direct knockout/knockdown (e.g., TALEN) can be used to inhibit T cell apoptosis. In embodiments, reducing the function or expression of downstream signals such as caspase8/3/fadd/caspase 10 and PRKC can be implemented to inhibit apoptosis. Dominant negative mutations have an altered gene product that acts antagonistically to the wild-type allele. These mutations usually result in an altered molecular function (often inactive) and are characterized by a dominant or semi-dominant phenotype. For example, the dominant negative form of the receptor may include one or more additions, deletions, or substitutions of the wide-type intracellular domain of the receptor such that a signaling pathway of the receptor may be blocked.

Embodiments relate to a method of expanding modified cells, the method comprising reducing or eliminating function or expression of the one or more molecules in the modified cells such that cell death of the modified cells induced by the one or more molecules is reduced as compared to the modified cells of which function of expression of the one or more molecules is not reduced or eliminated. In embodiments, reduced cell death achieves a similar effect (e.g, cell numbers) to those methods that directly increase cell numbers, which may also be considered as an expansion of the modified cells described herein.

Embodiments relate to a modified cell engineered to express an antigen binding molecule, wherein the expression and/or function of glucocorticoid receptor α (GRα) in the modified cell has been reduced or eliminated. Embodiments relate to a pharmaceutical composition comprising the population of the cells. Embodiments relate to a method of causing or eliciting T cell response in a subject in need thereof and/or treating a tumor of the subject, the method comprising administering an effective amount of the composition. For example, the modified cell comprises a disruption in an endogenous gene or the addition of an exogenous gene that is associated with a biosynthesis or transportation pathway of GRα.

In embodiments, the antigen binding molecule is the CAR, which comprises an antigen-binding domain, a transmembrane domain, and an intracellular signaling domain. In embodiments, the antigen-binding domain binds a tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL13Ra2, Mesothelin, IL11Ra, PSCA, PRSS21, VEGFR2, Lewis Y, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In embodiments, the intracellular signaling domain comprises a co-stimulatory signaling domain, or a primary signaling domain and a co-stimulatory signaling domain, wherein the co-stimulatory signaling domain comprises a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. Sequences may be found at US Patent Publication No: US20190216851, which is incorporated by reference herein in its entirely.

In embodiments, the antigen binding molecule is a modified TCR. In embodiments, the TCR is derived from spontaneously occurring tumor-specific T cells in patients. In embodiments, the TCR binds to a tumor antigen. In embodiments, the tumor antigen comprises CEA, gp100, MART-1, p53, MAGE-A3, or NY-ESO-1. In embodiments, the TCR comprises TCRγ and TCRδ Chains or TCRα and TCRβ chains, or a combination thereof.

In embodiments, the cell is an immune effector cell (e.g., a population of immune effector cells). For example, the immune effector cell is a T cell or an NK cell. In embodiments, the immune effector cell is a T cell. For example, the T cell is a CD4+ T cell, a CD8+ T cell, or a combination thereof. In embodiments, the cell is a human cell.

In embodiments, the modified cell comprises an inhibitor of the expression or function of one or more genes (e.g., GRα). In embodiments, the inhibitor is (1) a gene editing system targeted to one or more sites within the gene encoding the one or more genes or a corresponding regulatory elements; (2) nucleic acid encoding one or more components of a gene editing system of the one or more genes; or (3) combinations thereof.

In embodiments, the enhanced expression and/or function of the one or more genes is implemented by introducing a nucleic acid sequence of the one or more genes, which is present in the modified cell in a recombinant DNA construct, in an mRNA, or in a viral vector. In embodiments, the reduced expression and/or function of the one or more genes is implemented by introducing and overexpressing a nucleic acid sequence encoding GRβ, a dominant negative form of TNFR1, a dominant negative form of TNFR1, and/or a dominant negative form of Fas, and nucleic acid sequence is present in the modified cell in a recombinant DNA construct, in an mRNA, or in a viral vector. In embodiments, the nucleic acid is an mRNA, which is not integrated into the genome of the modified cell.

Embodiments relate to an isolated nucleic acid sequence encoding GRβ. In the modified cell, GRβ is overexpressed such that the modified cells may escape or reduce the possibilities of glucocorticoid-induced cell death.

In embodiments, the nucleic acid sequence is associated with an oxygen-dependent degradation domain. In embodiments, the oxygen-dependent degradation domain comprises a HIF1α domain that binds VHL. In embodiments, the nucleic acid sequence is regulated by a promoter comprising a binding site for a transcription modulator that modulates the expression and/or secretion of one or more therapeutic agents in the cell. In embodiments, the transcription modulator is or includes HIF1α, NFAT, FOXP3, and/or NFkB. In embodiments, the one or more therapeutic agents include one or more cytokines such as IL6, IL12, and IFNγ and/or one or more ECM molecule such as one or more ECM enzymes.

In embodiments, the antigen binding molecule is the CAR comprising an extracellular domain, a transmembrane domain, and an intracellular domain, and the extracellular domain binds an antigen.

In embodiments, the intracellular domain comprises a costimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Ra2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), and CD4.

In embodiments, the antigen binding molecule is a modified TCR. In embodiments, the TCR is derived from spontaneously occurring tumor-specific T cells in patients. In embodiments, the TCR binds a tumor antigen. In embodiments, the tumor antigen comprises CEA, gp100, MART-1, p53, MAGE-A3, or NY-ESO-1. In embodiments, the TCR comprises TCRγ and TCRδ chains or TCRα and TCRβ chains, or a combination thereof. In embodiments, the TCR comprises TCRγ and TCRδ chains or TCRα and TCRβ chains.

In embodiments, a T cell clone that expresses a TCR with a high affinity for the target antigen can be isolated. In embodiments, tumor-infiltrating lymphocytes (TILs) or peripheral blood mononuclear cells (PBMCs) can be cultured in the presence of antigen-presenting cells (APCs) pulsed with a peptide representing an epitope known to elicit a dominant T cell response when presented in the context of a defined HLA allele. High-affinity clones can be selected on the basis of MHC-peptide tetramer staining and/or the ability to recognize and lyse target cells pulsed with low titrated concentrations of cognate peptide antigen. After the clone is selected, the TCRα and TCRβ chains or TCRγ and TCRδ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle (e.g., a gammaretrovirus or lentivirus) can be generated and tested for functionality (antigen specificity and functional avidity) and used to produce a clinical batch of the vector. An aliquot of the final product can then be used to transduce the target T cell population (generally purified from patient PBMCs), and expanded before infusion into the patient. Various methods can be implemented to obtain genes encoding tumor-reactive TCR. More information is provided in Kershaw et al., Clin Transl Immunology. 2014 May; 3(5): e16, which is incorporated herein by reference in its entirety.

In embodiments, specific TCR can be derived from spontaneously occurring tumor-specific T cells in patients. Antigens included in this category include the melanocyte differentiation antigens MART-1 and gp100, as well as the MAGE antigens and NY-ESO-1, with expression in a broader range of cancers. TCRs specific for viral-associated malignancies can also be isolated as long as viral proteins are expressed by transformed cells. Malignancies in this category include liver and cervical cancer, associated with hepatitis and papillomaviruses, and Epstein-Barr virus-associated malignancies. In embodiments, target antigens of the TCR may include CEA (e.g., for colorectal cancer), gp100, MART-1, p53 (e.g., for Melanoma), MAGE-A3 (e.g., Melanoma, esophageal and synovial sarcoma), NY-ESO-1 (e.g., for Melanoma and sarcoma as well as Multiple myelomas).

In embodiments, expression of the one or more polynucleotides described herein, may be regulated by an inducible expression system. The inducible expression system allows for a temporal and spatial controlled activation and/or expression of genes. For example, tetracycline-controlled transcriptional activation is a method of inducible gene expression where transcription is reversibly turned on or off in the presence of the antibiotic tetracycline or one of its derivatives (e.g., doxycycline). For example, an inducible suicide gene expression system allows for a temporal and spatial controlled activation and/or expression of a suicide gene, which causes a cell to kill itself through apoptosis.

In embodiments, the modified cells described herein comprise a nucleic acid sequence encoding a reverse tetracycline transactivator (rtTA). In embodiments, expression of the one or more molecules is regulated by the rtTA, such that the one or more polynucleotides are expressed in the presence of tetracycline. In embodiments, a concentration of tetracycline in the cell culture medium is not less than about 2 μg/ml. In embodiments, the tetracycline is selected from the group consisting of tetracycline, demeclocycline, meclocycline, doxycycline, lymecycline, methacycline, minocycline, oxytetracycline, rolitetracycline, and chlortetracycline. In embodiments, the tetracycline is doxycycline.

In embodiments, the inducible suicide system is an HSV-TK system or an inducible caspase-9 system. In embodiments, the modified cells comprise a nucleic acid sequence encoding a suicide gene, such that when the modified cells are in the presence of a nucleoside analog, the suicide gene is expressed which renders the nucleoside analog cytotoxic to the modified cells. In embodiments, the suicide gene is selected from the group consisting of thymidine kinase of herpes simplex virus, thymidine kinase of varicella zoster virus, and bacterial cytosine deaminase. In embodiments, the suicide gene is the thymidine kinase of the herpes simplex virus. In embodiments, the nucleoside analog is selected from the group consisting of ganciclovir, acyclovir, buciclovir, famciclovir, penciclovir, valciclovir, trifluorothymidine, 1-[2-deoxy, 2-fluoro, beta-D-arabino furanosyl]-5-iodouracil, ara-A, araT 1-beta-D-arabinofuranoxyl thymine, 5-ethyl-2′-deoxyuridine, 5-iodo-5′-amino-2,5′-dideoxyuridine, idoxuridine, AZT, AIU, dideoxycytidine, and AraC. In embodiments, the nucleoside analogue is ganciclovir.

In embodiments, the antigen binding molecule is CAR, which comprises an antigen-binding domain, a transmembrane domain, and an intracellular signaling domain. In embodiments, the antigen-binding domain binds to a tumor antigen is selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL13Ra2, Mesothelin, IL11Ra, PSCA, PRSS21, VEGFR2, Lewis Y, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In embodiments, the intracellular signaling domain comprises a co-stimulatory signaling domain, or a primary signaling domain and a co-stimulatory signaling domain, wherein the co-stimulatory signaling domain comprises a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

Embodiments relate to a method or use of the polynucleotide described herein The method of use includes: providing a viral particle (e.g., AAV, lentivirus, or their variants) comprising a vector genome, the vector genome comprising the polynucleotide, wherein the polynucleotide is operably linked to an expression control element conferring transcription of the polynucleotide and administering an amount of the viral particle to the subject such that the polynucleotide is expressed in the subject. In embodiments, the AAV preparation may include AAV vector particles, empty capsids, and host cell impurities, thereby providing an AAV product substantially free of AAV empty capsids. More information on the administration and preparation of the viral particle may be found in the U.S. Pat. No. 9,840,719 and Milani et al., Sci. Transl. Med. 11, eaav7325 (2019) 22 May 2019, which are incorporated herein by reference.

Embodiments relate to a pharmaceutical composition comprising the population of the cells described herein. Embodiments relate to a method of causing, inducing, or eliciting T cell response in a subject in need thereof and/or treating a tumor of the subject, the method comprising administering an effective amount of the composition. Embodiments relate to a method of delivering one or more therapeutic agents, the method comprising administering an effective amount of the composition. In embodiments, the one or more therapeutic agents include one or more cytokines such as IL6, IL12, and IFNγ and/or one or more ECM molecules such as one or more ECM enzymes.

In embodiments, one or more polynucleotides are present in the modified cell in a recombinant DNA construct, in an mRNA, or in a viral vector. In embodiments, the one or more polynucleotides are mRNAs, which are not integrated into the genome of the modified cell. In embodiments, the one or more polynucleotides are associated with an oxygen-dependent degradation domain. In embodiments, the oxygen-dependent degradation domain comprises a HIF1α domain that binds VHL. In embodiments, expression of the one or more polynucleotides is regulated by a promoter comprising a binding site for a transcription modulator that modulates the expression and/or secretion of one or more therapeutic agents in the cell. In embodiments, the transcription modulator is or includes HIF1α, NFAT, FOXP3, and/or NFkB. In embodiments, the one or more therapeutic agents include one or more cytokines such as IL6, IL12, and IFNγ and/or one or more ECM molecules such as one or more ECM enzymes.

In embodiments, the expression the of one or more polynucleotides is regulated by NFAT such that the extracellular vesicle (EV) is assembled in response to the activation of the modified cell. Embodiments relate to a polynucleotide comprising a binding site of a transcription modulator (e.g., NFAT) and encoding one or more proteins assembling the EV and one or more therapeutic agents. In embodiments, the one or more therapeutic agents include one or more cytokines such as IL6, IL12, and/or IFNγ or one or more ECM molecules such as one or more ECM enzymes.

In embodiments, the one or more proteins are self-assembling proteins. In embodiments, the one or more proteins that direct their release through vesicles as a luminal membrane-bound protein is chosen from the group consisting of the retroviral group specific antigen, retroviral group specific antigen variations, the influenza MI protein, the ARRDCI protein, the ARC protein, the Ebola virus VP40 protein, and the M proteins of vesicular stomatitis virus. In embodiments, the one or more proteins comprise an Arc protein, and the one or more polynucleotides comprise a nucleic acid encoding one or more therapeutic agents. In embodiments, the one or more therapeutic agents include one or more cytokines such as IL6, IL12, and IFNγ and/or one or more ECM molecules such as one or more ECM enzymes. More information on ARC based EV may be found in PCT Patent Publication Nos: WO2019091964 and WO2019118497 as well as Pastuzyn et al., Cell. 2018 Jan. 11; 172(1-2): 275-288.e18, which are herein incorporated by reference.

Embodiments relate to a modified cell engineered to express an antigen binding molecule, wherein the expression and/or function of one or more molecules in the modified cell has been enhanced, and wherein the one or more molecules degrade one or more ECM molecules. In embodiments, the modified cell comprises a disruption in an endogenous gene or an addition of an exogenous gene associated with the biosynthesis or transportation pathway of the one or more molecules. Embodiments relate to a method or use of one or more polynucleotides, the method comprising providing a viral particle (e.g., AAV, lentivirus or their variants) comprising a vector genome, which comprises a polynucleotide encoding the one more molecules, and a polynucleotide encoding a binding molecule, the polynucleotide, as described above, operably linked to an expression control element conferring transcription of the polynucleotides; and administering an amount of the viral particle to a subject such that the polynucleotide is expressed in the subject, wherein the one or more molecules are overexpressed in cancer cells, associated with recruitment of immune cells, and/or associated with autoimmunity. In embodiments, the viral particle comprises an AAV preparation which can include AAV vector particles, empty capsids, and host cell impurities, thereby providing an AAV product substantially free of AAV empty capsids.

Embodiments relate to a pharmaceutical composition comprising a population of cells (e.g., CAR T cells). Embodiments relate to a method of causing, inducing, or eliciting T cell response in a subject in need thereof and/or treating a tumor of the subject, the method comprising administering an effective amount of the pharmaceutical composition to the subject. Embodiments relate to an isolated nucleic acid sequence encoding one or more molecules that are overexpressed in cancer cells, associated with recruitment of immune cells, and/or associated with autoimmunity.

In embodiments, the expression of the one or more molecules that degrade one or more ECM molecules can be regulated by an inducible expression system. The inducible expression system allows for a temporal and spatial controlled activation and/or expression of genes. For example, Tetracycline-controlled transcriptional activation is a method of inducible gene expression where transcription is reversibly turned on or off in the presence of the antibiotic tetracycline or one of its derivatives (e.g., doxycycline). For example, an inducible suicide gene expression system allows for a temporal and spatial controlled activation and/or expression of a suicide gene, which causes a cell to kill itself through apoptosis.

In embodiments, the polynucleotide described herein, for example, the polynucleotides encoding the CARs and ECM enzymes may integrate into the genome of the modified cell, and descendants of the modified cell will also express the polynucleotide, resulting in a stably transfected modified cell. In embodiments, the modified cell may express the polynucleotide encoding the CAR, but the polynucleotide does not integrate into the genome of the modified cell such that the modified cell expresses the transiently transfected polynucleotide for a finite period of time (e.g., several days), after which the polynucleotide is lost through cell division or other factors. For example, the polynucleotide is present in the modified cell in a recombinant DNA construct, in an mRNA, or in a viral vector, and/or the polynucleotide is an mRNA, which is not integrated into the genome of the modified cell.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples but rather should be construed to encompass any and all variations which become evident as a result of the teaching provided herein.

### EXAMPLES

Lentiviral vectors that encode a CAR were generated (see Chimeric Receptors Containing CD137 Signal Transduction Domains Mediate Enhanced Survival of T Cells and Increased Antileukemic Efficacy In Vivo Molecular Therapy vol. 17 no. 8, 1453-1464 August 2009 incorporated herein by reference) and were introduced into human T cells. Primary T cells were obtained from patients. The primary T cells were transduced with lentiviral vectors to obtain modified T cells. Flow cytometry was performed and analyzed to confirm the expression of CARs in primary T cells. Techniques related to cell cultures, construction of lentiviral vectors, and flow cytometry can be found in the Control of large, established tumor xenografts with genetically retargeted human T cells containing CD28 and CD137 domains. 3360-3365 PNAS Mar. 3, 2009, vol. 106 no. 9, which is incorporated herein by reference.

Lentiviral vectors encoding GCC CAR and CK as well as NE that are regulated by the IL2 minimal promoter and NFAT enhancer (See Construct 102) were transferred into T cells to obtain CAR T cells overexpressing CK and NE. The NFAT enhancer includes repeats of NFAT binding sites and enhances translation of CK and NE. The signal peptide (SP) of NE was used to direct NE expression on the T cells. While CAR was expressed normally on the T cells, little expression of NE was measured by flow cytometry. Several other signal peptides were tested to overexpress NE, and the expression of NE was measured. The expression of NE directed by IL2 SP (SEQ ID NO: 42 or 43) was significantly higher than that of other constructs, such as TPA and IFNγ. Further, lentiviral vectors encoding GCC CAR as well as CK, NE, and MMP7 that are regulated by IL2 minimal promoter and NFAT enhancer (See Construct 108) were transferred into T cells to obtain CAR T cells overexpressing CK, NE, and MMP7. The constructs were introduced into T cells, and the functionalities of these T cells were then measured.

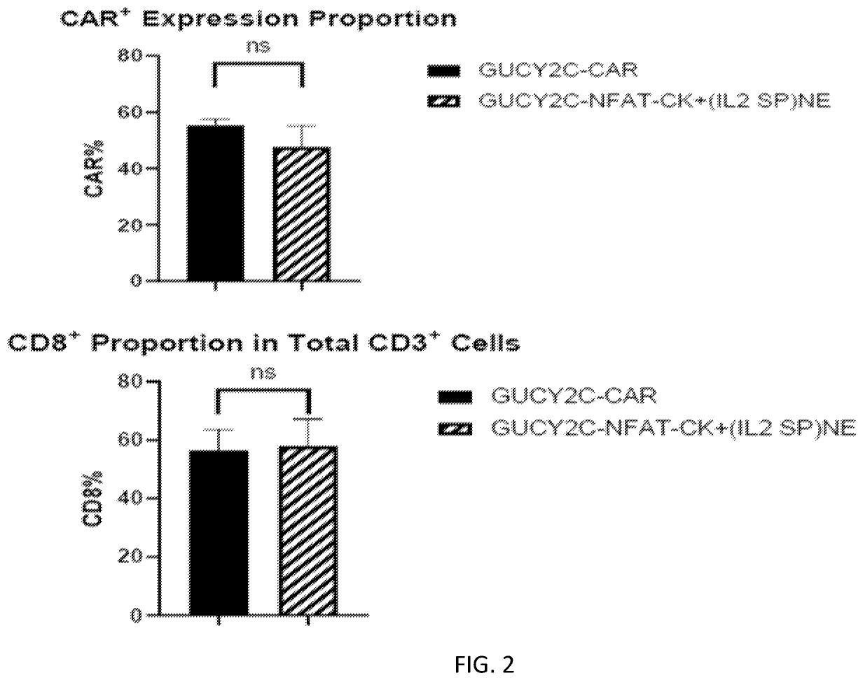

FIG. 2 shows the expression of CAR and NE. Lentiviral vectors encoding GCC CAR and CK, as well as NE (CK+(IL2 SP) NE), were transferred into T cells, respectively. The T cells were then cultured for 8 days, and assays were conducted to measure the amounts of the expression of CAR. As shown in FIG. 2, the amounts of the expression of GCC CAR in cells including lentiviral vectors encoding GCC CAR (GCC CAR T cells) and cells including lentiviral vectors encoding GCC CAR and CK+(IL2 SP) NE (GCC CAR CK+NE cells) were similar.

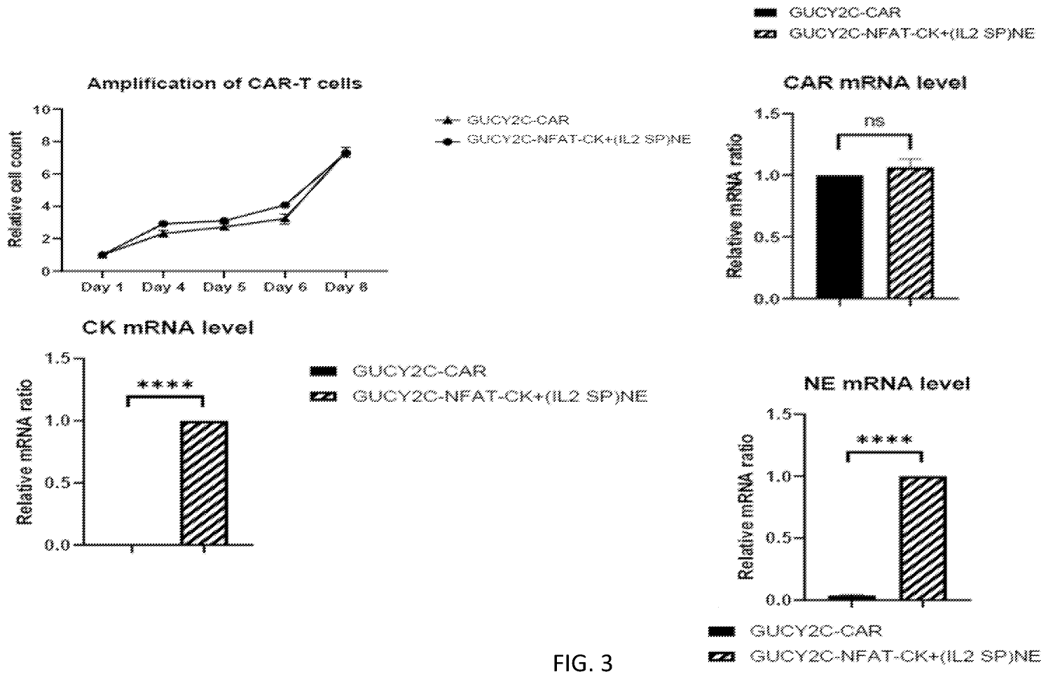

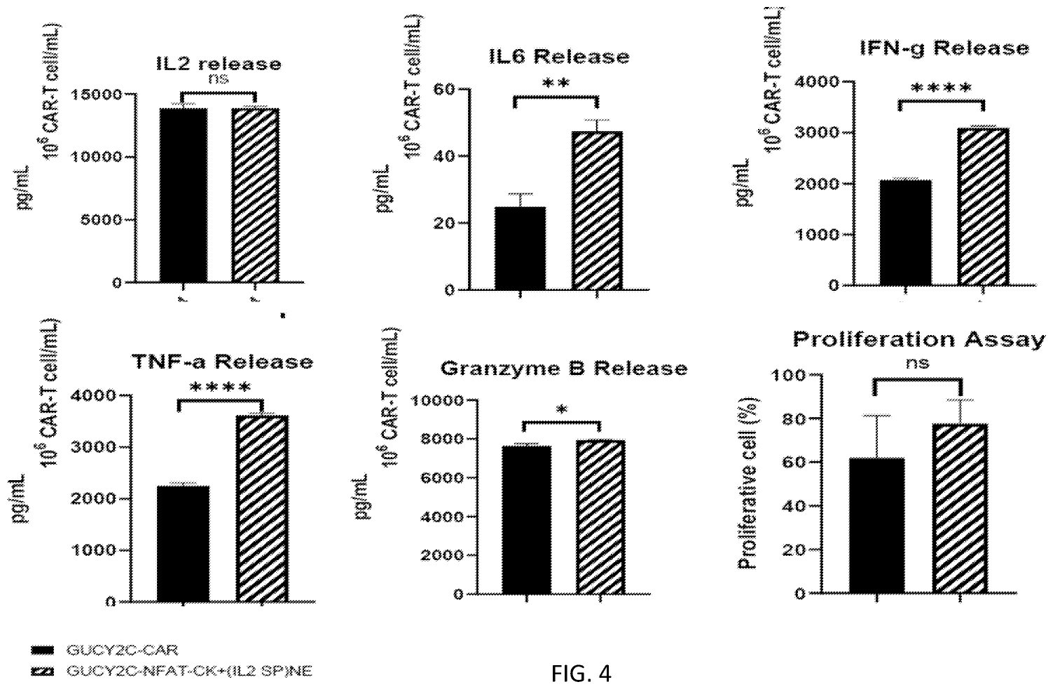

FIG. 3 shows amplification of CAR T cells and mRNA levels of GCC CAR, CK, and NE in the CAR T cells. FIG. 4 shows levels of cytokine release and proliferation between GCC CAR T cells and GCC CAR CK+NE cells after these cells were co-cultured with T84 cells, which are substrate cells of GCC CAR. These results demonstrate that GCC CAR CK+NE cells cause similar or more T cell responses than GCC CAR T cells.

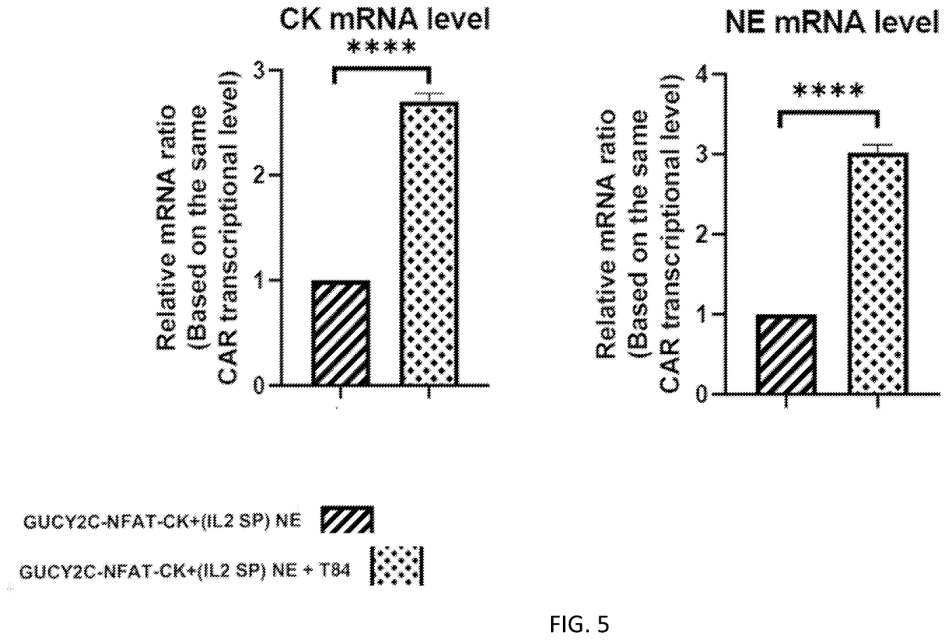

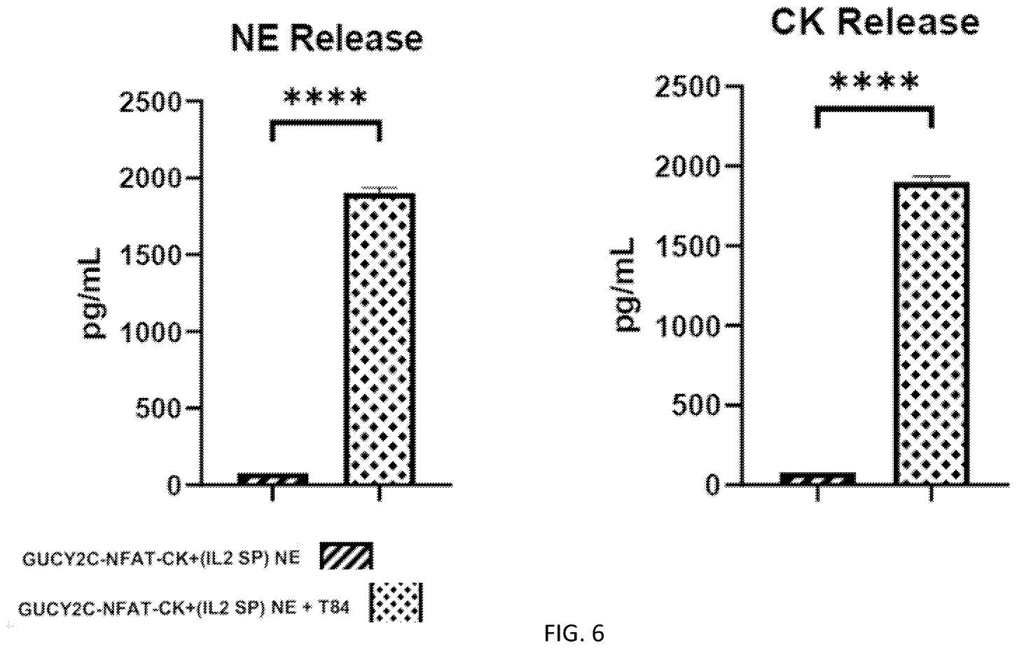

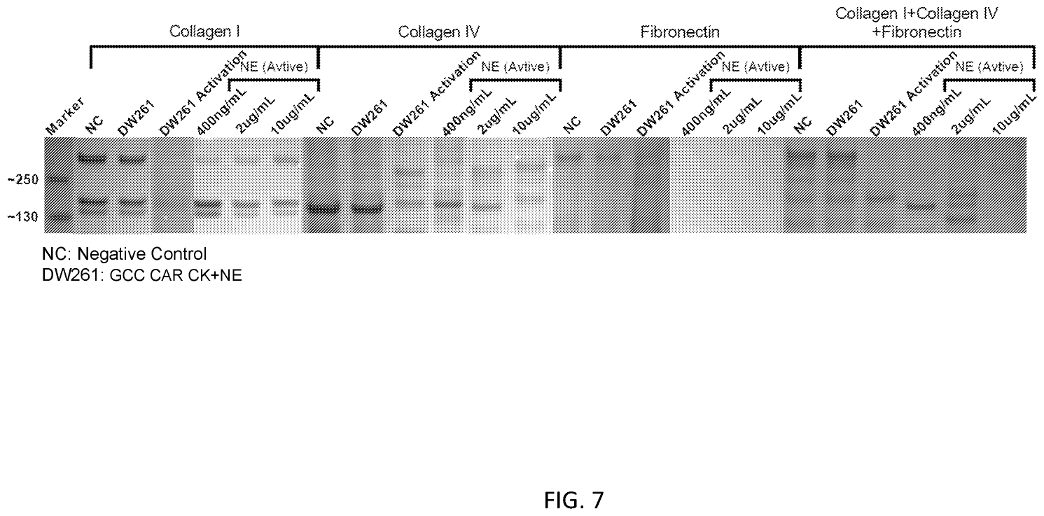

FIG. 5 shows levels of mRNA of NE and CK in GCC CAR T cells cultured alone and co-cultured with T84, respectively. FIG. 6 shows NE and CK released by GCC CAR T cells cultured alone and co-cultured with T84, respectively. These results demonstrate that transcription and expression of CK and NE in GCC CAR CK+NE cells increased significantly when these cells were co-cultured with the substrate cells. FIG. 7 shows results of Western Blot of ECM molecules degraded by various solutions. GCC CAR CK+NE cells were cultured alone and co-cultured with T84, respectively. Culture media were obtained from these two cell cultures (DW261 and DW261 Activation) and mixed with ECM molecules (collagen I, collagen IV, fibronectin, and a mixture of these three molecules). Western Blot was conducted to measure the ability of degradation of the ECM molecules. These results demonstrate that NE released by GCC CAR CK+NE cells can degrade ECM molecules when GCC CAR CK+NE Cells are co-cultured with the substrate cells. Further, these results showed that the NFAT successfully regulates transcription and expression of CK and NE in GCC CAR CK+NE cells, therefore enhancing the safety of GCC CAR CK+NE Cells in its cellular therapy.

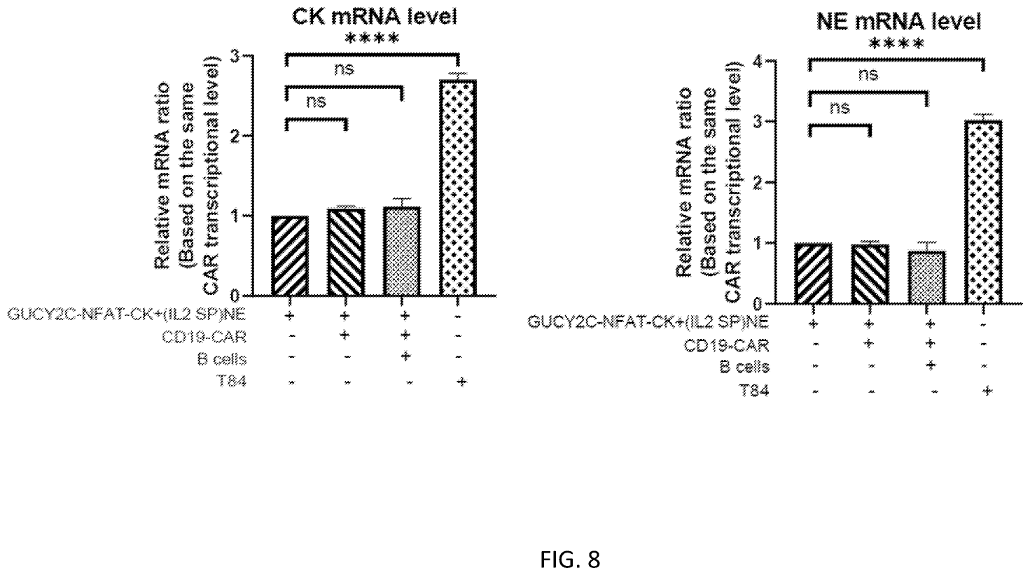

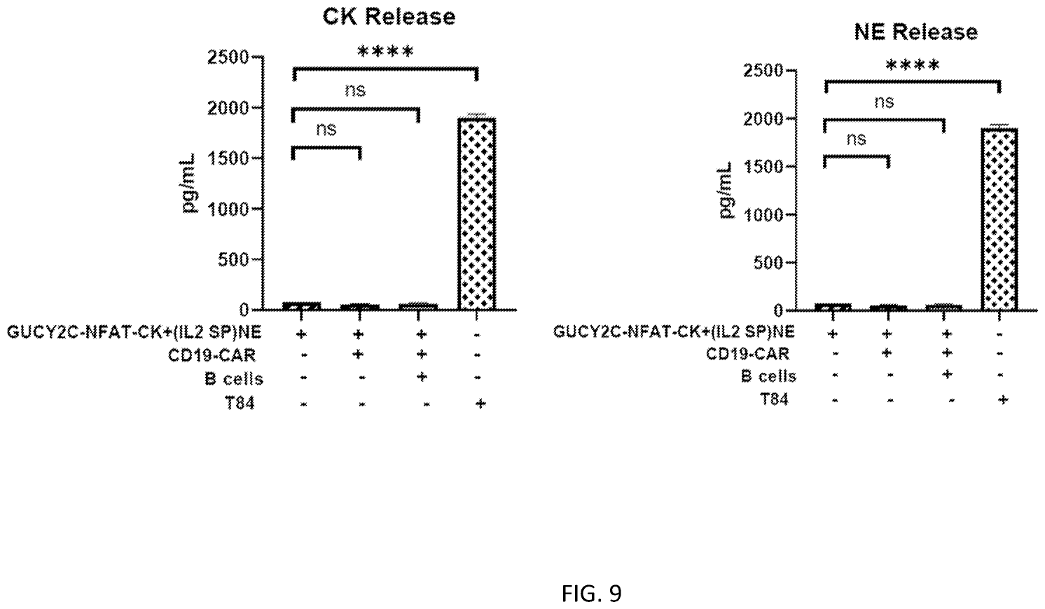

FIG. 8 shows levels of mRNA GCC CAR CK+NE cells in the CoupledCAR® system. FIG. 9 shows NE and CK released by GCC CAR CK+NE in the CoupledCAR® system. The CoupledCAR® system is described in PCT Publication Nos: WO2020106843 and WO2020146743, as well as US Publication NO: US20210100841. These results demonstrate that GCC CAR CK+NE cells function normally in the CoupledCAR® system.

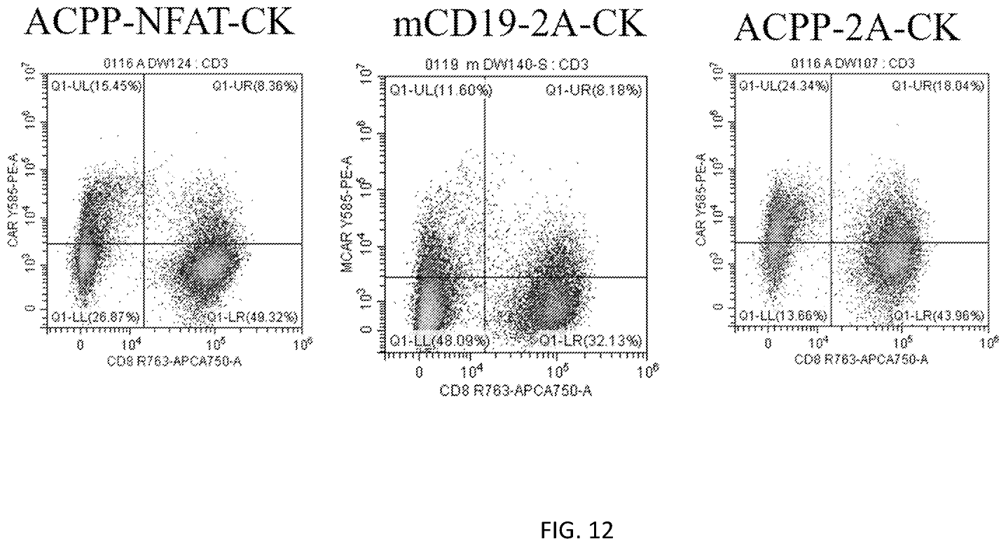

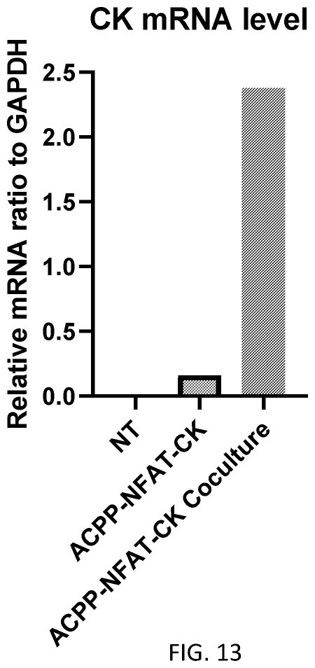

FIG. 12 shows flow cytometry of expression of CK and CD19 CAR or ACPP CAR on T cells. FIG. 13 shows mRNA levels of CK in non-transduced T cells (NT) (Group 1), T cells expressing CK and ACPP CAR (Group 2), and T cells expressing CK and ACPP CAR and being co-cultured with substrate cells (Group 3). As shown in FIG. 13, the level of mRNA of CK in the cells of Group 3 is significantly higher than in the cells of the other two groups.

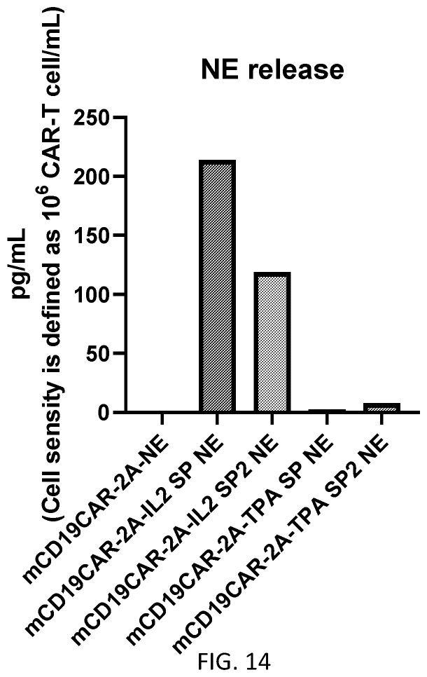

FIG. 14 shows comparisons of NE released by T cells transduced with CARs and NE driven by different SPs. T cells were transduced with lentiviral vectors encoding CAR and NE, including the original SP of NE. While CARs were expressed on these T cells normally, little NE was detected in the supernatant of culture media. The original SP of NE was replaced by various SP domains in lentiviral vectors, which were transduced into T cells. NE released by these T cells was measured. Among these SPs, the SP of IL2 caused significantly more NE release than other SPs, and the short version of the SP of IL2 (SEQ ID NO: 42) causes more NE to release than other SPs, including the entire length of the SP of IL2. For example, as shown in FIG. 14, T cells transduced with the vectors encoding NE and the SP of IL2 released significantly more NE than other T cells.

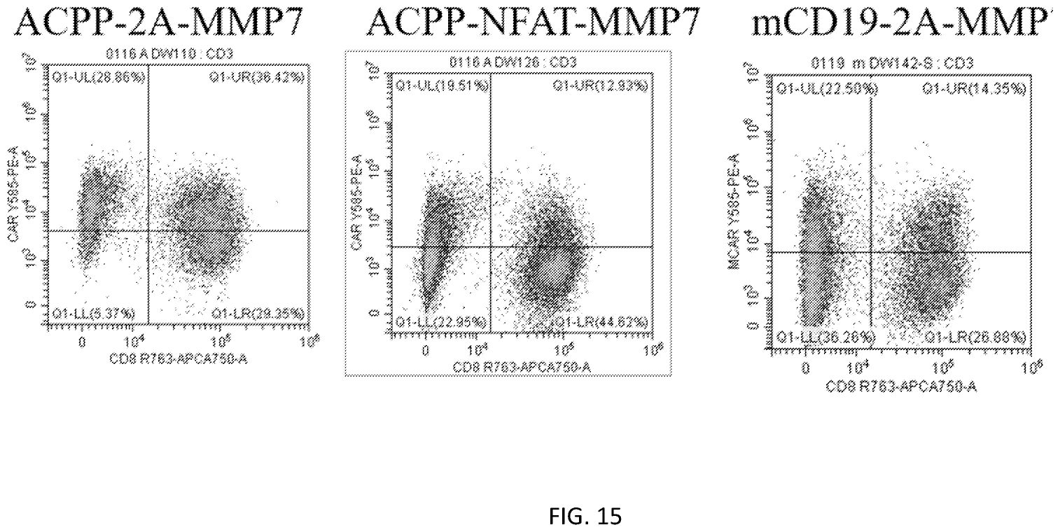

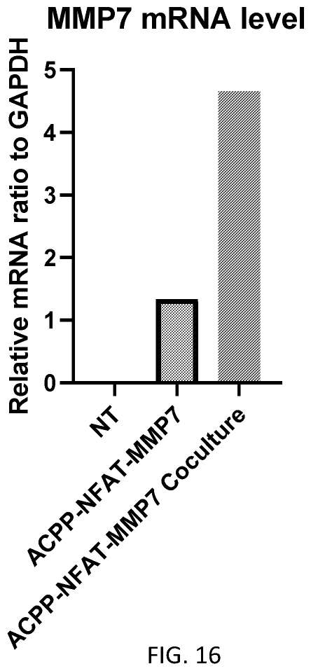

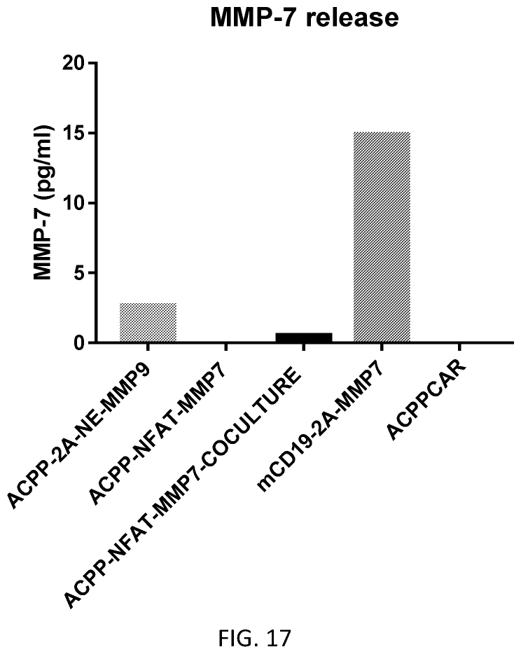

FIG. 15 shows flow cytometry of expression of MMP7 and CD19 CAR or ACPP CAR on T cells. FIGS. 16 and 17 show mRNA levels or protein levels of CK in the non-transduced T cells (NT) (Group 1), T cells expressing CK and ACPP CAR (Group 2), and T cells expressing CK and ACPP CAR and being co-cultured with substrate cells (Group 3). For example, as shown in FIG. 17, the level of mRNA of CK in the cells of Group 3 is significantly higher than in cells of the other two groups. FIG. 17 shows CK released by the cells of these three groups.

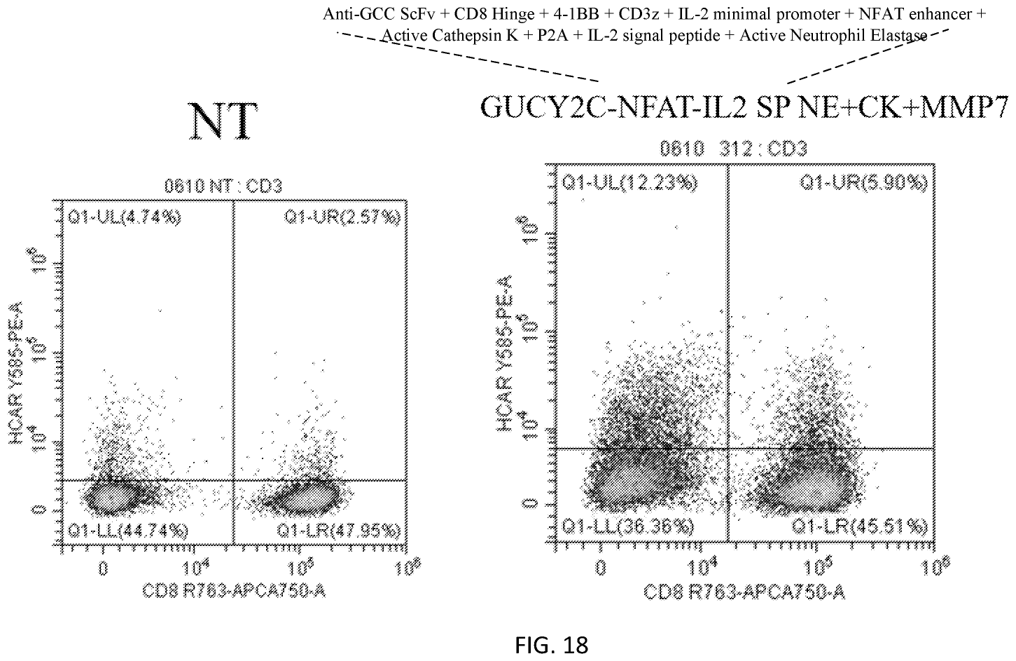

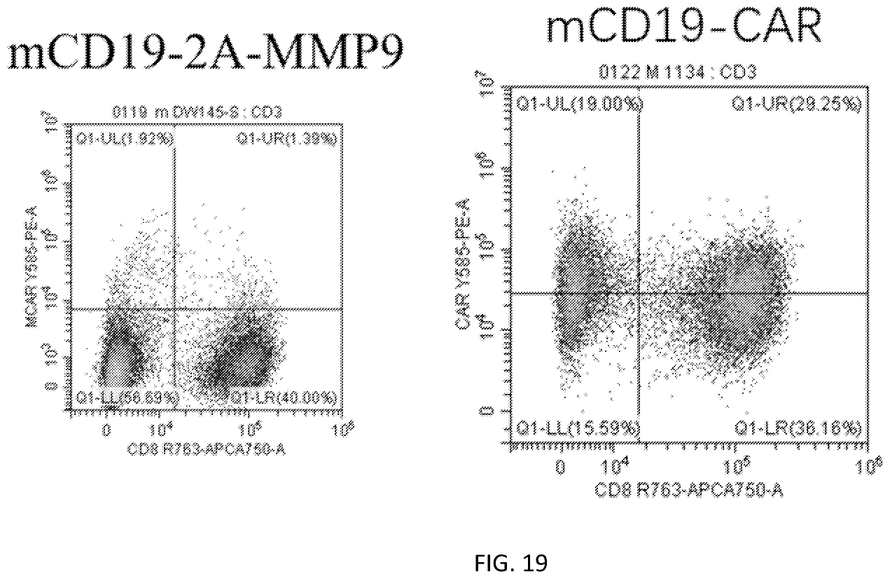

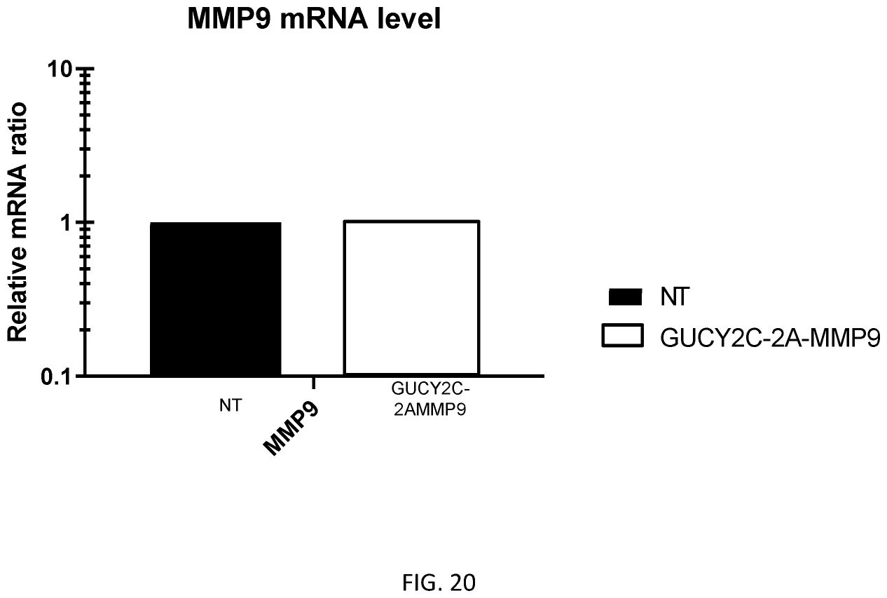

FIG. 18 shows flow cytometry of expression of NE, CK, MMP7, and GUCY2C CAR on T cells. However, not all enzymes degrading ECM molecules can be co-expressed with CARs. For example, as shown in FIGS. 19 and 20, while CAR was expressed normally on T cells, flow cytometry and RT-qPCR assays showed little expression of MMP9 directed by its own SP on these T cells. FIG. 19 shows flow cytometry of the expression of MMP9 and CD19 CAR on T cells. FIG. 20 shows the levels of mRNA of MMP9 on T cells expressing GUCY2C CAR.

These results show that Construct 102 of FIG. 1 was packaged into lentiviral vectors and successfully introduced into T cells. These T cells degraded ECM molecules in the conventional CAR and CoupledCAR® system when activated. The sequences used in the Examples can be found in Table 2 and in PCT Publication Nos: WO2020106843 and WO2020146743, as well as US Publication NO: US20210100841.

TABLE 2
Sequences

| Component | SEQ ID NO: |
|---|---|
| Anti-GCC ScFv | 1 |
| CD8 Hinge | 2 |
| 4-1BB | 3 |
| CD3z | 4 |
| IL2 minimal promoter | 5 |
| NFAT enhancer (Repeats of NFAT binding sites) | 6 |
| Active Cathepsin K | 7 |
| P2A | 8 |
| IL2 signal peptide | 9 |
| Active Neutrophil Elastase | 10 |
| NE + IL2SP (1) | 11 |
| NE + IL2SP (2) | 12 |
| MMP13 | 13 |
| MMP2 | 14 |
| MMP9 | 15 |
| MMP7 | 16 |
| MMP8 | 17 |
| MMP3 | 18 |
| Plasmin | 19 |
| PLAT(tPA) | 20 |
| PLAU(uPA) | 21 |
| Cathepsin B | 22 |
| MMP1 | 23 |
| MMP10 | 24 |
| MMP11 | 25 |
| MMP12 | 26 |
| MMP14 | 27 |
| MMP15 | 28 |
| MMP16 | 29 |
| MMP17 | 30 |
| MMP24 | 31 |
| MMP25 | 32 |
| MMP26 | 33 |
| ADAMTS-1 | 34 |
| ADAMTS-4 | 35 |
| ADAMTS-5 | 36 |
| ADAMTS-8 | 37 |
| ADAMTS-9 | 38 |
| ADAMTS-15 | 39 |
| ADAMTS-20 | 40 |
| heparanase | 41 |
| IL2 SP | 42 |
| IL2 SP2 | 43 |
| TPA SP | 44 |
| TPA SP2 | 45 |
| WT SP | 46 |
| MMP9 | 47 |
| NFAT enhancer-IL2 minimal promoter -kozak-CK | 48 |
| (IL2SP-NE)-P2A-MMP7 | 49 |
| (IL2SP-NE)-P2A-MMP7 | 50 |
| CK-P2A-(IL2SP-NE) | 51 |
| NFAT enhancer-IL2 minimal promoter -kozak-CK-P2A- | 52 |
| (IL2SP-NE) | |
| CK-P2A-(IL2SP-NE)-P2A-MMP7 | 53 |
| NFAT enhancer-IL2 minimal promoter -kozak-CK-P2A- | 54 |
| (IL2SP-NE)-P2A-MMP7 | |
| Mini-ODD 1 (VHL binding) | 55 |
| Mini-ODD 2 (VHL binding) | 56 |
| Mini-ODD 3 (VHL binding) | 57 |

1. A modified cell engineered to express and secrete one or more agents targeting one or more extracellular matrix (ECM) molecules, the modified cell comprising a polynucleotide encoding a chimeric antigen receptor (CAR) and a polynucleotide encoding one or more agents, and the one or more agents comprising at least one of cathepsin K (CK), neutrophil elastase (NE), and matrix metalloproteinase-7 (MMP7).

2. The modified cell of claim 1, wherein the polynucleotide encoding one or more agents comprise SEQ ID NO: 48, 50, 52, or 54.

3. The modified cell of claim 1, wherein the polynucleotide encoding one or more agents comprise a nucleic acid encoding SEQ ID NO: 49, 51, or 53.

4. The modified cell of claim 1, wherein the polynucleotide encoding the one or more agents further comprise a nucleic acid comprising a nuclear factor of activated T-cells (NFAT) binding site, and a nucleic acid encoding oxygen-dependent degradation (ODD) domain of HIF1α, wherein the nucleic acid encoding the agent is flanked by the nucleic acid comprising the NFAT binding site and the nucleic acid encoding the ODD domain of HIF1α.

5. The modified cell of claim 4, wherein the ODD domain of HIF1α comprises SEQ ID NO: 55, 56, or 57.

6. The modified cell of claim 1, wherein the nucleic acid comprising the NFAT binding site comprises one or more NFAT binding sites followed by the minimal IL2 promoter.

7. The modified cell of claim 1, wherein the expression of the one or more agents is regulated by HIF1α, NFAT, forkhead box P3 (FOXP3), or nuclear factor kappa B (NF-κB).

8. The modified cell of claim 1, wherein the one or more agents comprise CK.

9. The modified cell of claim 1, wherein the polynucleotide encoding the one or more agents comprise (1) SEQ ID NOs: 5 and 6 and (2) a nucleotide encoding SEQ ID NO: 7.

10. The modified cell of claim 1, wherein the polynucleotide encoding the one or more agents comprise (1) SEQ ID NO: 48, 52, or 54 or (2) a nucleic acid encoding SEQ ID NO: 51 or 53.

11. The modified cell of claim 1, wherein the one or more agents comprise NE.

12. The modified cell of claim 1, wherein the polynucleotide encoding the one or more agents comprise a nucleic acid encoding a signaling peptide of IL2 and a nucleic acid encoding NE.

13. The modified cell claim 1, wherein the polynucleotide encoding the one or more agents comprise:
a nucleic acid encoding SEQ ID NO: 10 and a nucleic acid encoding SEQ ID NO: 42 or 43;
SEQ ID NO: 11 or 12; or
SEQ ID NO: 9 and a nucleic acid encoding SEQ ID NO: 10.

14. The modified cell of claim 1, wherein the one or more agents comprise MMP7.

15. The modified cell of claim 1, wherein the one or more agents comprise CK and NE.

16. (canceled)

17. The modified cell of claim 1, wherein the one or more agents comprise CK, NE, and MMP7.

18. (canceled)

19. The modified cell of claim 1, wherein the one or more ECM molecules comprise at least one of collagen I, collagen III, collagen VI, collagen IV, and fibronectin.

20. The modified cell of claim 1, wherein the CAR comprises an antigen-binding domain, a transmembrane domain, and an intracellular signaling domain.

21. The modified cell of claim 20, wherein the antigen binding domain binds a tumor antigen, comprising TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL13Ra2, Mesothelin, IL11Ra, PSCA, PRSS21, VEGFR2, Lewis Y, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, T_{EM}1/CD248, T_{EM}7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, or IGLL1.

22. The modified cell of claim 20 or 21, wherein the intracellular signaling domain further comprises a co-stimulatory signaling domain, and wherein the co-stimulatory signaling domain comprises a functional signaling domain of a protein comprising CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D.

23. The modified cell of claim 1, wherein the modified cell comprises a dominant negative form of an inhibitory immune checkpoint molecule or a receptor thereof, and the inhibitory immune checkpoint molecule comprises programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), or CD 160.

24. The modified cell of claim 23, wherein the modified cell has a reduced expression of endogenous TRAC gene.

25. A method of inducing a T cell response, the method comprising:
contacting a cell comprising a tumor antigen with the modified cell claim 1, wherein the CAR binds the tumor antigen, and the T cell response comprises release of IFNγ.
